SEQUENCE LISTING (10 sequences)

SEQ ID NO: 1 - DNA, 972 bp, Glycine max
atggagaagaaggagatggctgtgaaaactgaggatgcggttgggtcctcttctttcccttgttataattattcaaacctttatccattctcgaacgcgtttgatttctctgaggttgaaaagagctctttagggtttatggagttactgggtgtgcaggactatagtcctctgcttgagttgcctcaactttcaactgtgtccgtgcaatctcatcatcactctacagttacggttccttctgataacgggaaagagtgttctgaggtgttgaaccaccagcctgccactccaaactcttcttccatttcatccgcgtccactgatgcagtcaatgatgaacagaagaagactctagaccaagcagaagaagatgatgatgatgatgatgaaggacgacacaagactaagaaacagttgaagcccaagaagacaaatcagaagagacagagagaaccaagattcgcgttcatgacgaaaagcgaggtcgatcatctggaagatgggtacagatggagaaagtacggtcaaaaggccgtgaaaaatagcccctttcccaggagctactatcgttgcaccagtgtttcatgtaatgtgaagaaacgtgtggagagatctttcactgatccaagcgttgtagtgacaacctatgaaggccaacacacacatcccagcccagttatgcctcgctcagttgtctcttctggatacgccaacaactttgcttcagttttgccactaggaaactacttatcccagtatcagcagcagcaccatcaccaccaacaacaaaagctacttgtcaacacattgtcctctttgggttttccttacaatgattcttcatctccaaagaacgctgtttttattcaagagagacggctttgtagtaatcaagggacgaatgcgtttctgagggaccatggacttcttcaagatgttgttccttcacacatgttgaaagaagagtag

SEQ ID NO: 2 - protein, 323 aa, Glycine max
MEKKEMAVKTEDAVGSSSFPCYNYSNLYPFSNAFDFSEVEKSSLGFMELLGVQDYSPLLELPQLSTVSVQSHHHSTVTVPSDNGKECSEVLNHQPATPNSSSISSASTDAVNDEQKKTLDQAEEDDDDDDEGRHKTKKQLKPKKTNQKRQREPRFAFMTKSEVDHLEDGYRWRKYGQKAVKNSPFPRSYYRCTSVSCNVKKRVERSFTDPSVVVTTYEGQHTHPSPVMPRSVVSSGYANNFASVLPLGNYLSQYQQQHHHHQQQKLLVNTLSSLGFPYNDSSSPKNAVFIQERRLCSNQGTNAFLRDHGLLQDVVPSHMLKEE

SEQ ID NO: 3 - DNA, 20 bp, Artificial
  Primer
aacctcctcctcatcgtact

SEQ ID NO: 4 - DNA, 20 bp, Artificial
  Primer
gacagcatcagccatgttca

SEQ ID NO: 5 - DNA, 20 bp, Artificial
  Primer
atggagaagaaggagatggc

SEQ ID NO: 6 - DNA, 20 bp, Artificial
  Primer
ctactcttctttcaacatgt

SEQ ID NO: 7 - DNA, 30 bp, Artificial
  Primer
atcagaattcatggagaagaaggagatggc

SEQ ID NO: 8 - DNA, 30 bp, Artificial
  Primer
gatgctgcagctactcttctttcaacatgt

SEQ ID NO: 9 - DNA, 30 bp, Artificial
  Primer
atcaggatccatgacagtagatctggtagg

SEQ ID NO: 10 - DNA, 31 bp, Artificial
  Primer
gatgggtacctgacagtagatctggtaggtg

(19) United States
(12) Patent Application Publication
(10) Pub. No.: US 2010/0263093 A1
(43) Pub. Date: Oct. 14, 2010

(54) TRANSGENIC PLANTS AND MODULATORS FOR IMPROVED STRESS TOLERANCE

(75) Inventors: Shouyi Chen, Beijing (CN); Jingsong Zhang, Beijing (CN); Qiyun Zhou, Beijing (CN); Aiguo Tian, Beijing (CN); Sijie He, Beijing (CN); Baoxin Du, Beijing (CN)

Correspondence Address: Womble Carlyle Sandridge & Rice PLLC;Syngenta Biotechnology, Attention: Patent Docketing, P.O. Box 7037, Atlanta, GA, 30357-0037, US

(73) Assignee: INSTITUTE OF GENETICS AND DEVELOPMENT BIOLOGY, CHI, Beijing (CN)

(21) Appl. No.: 12/740,317

(22) PCT Filed: Oct. 29, 2008
(86) PCT No.: PCT/IB2008/054502

§ 371 Date: May 28, 2010

(30) Foreign Application Priority Data
Oct. 29, 2007 (CN) 200710176476.0

(51) Int. Cl. A01H 5/00; C12N 15/11; C12N 15/63; C12N 5/10; C07K 14/435; C07K 16/18; C12N 15/82; C12Q 1/68; G01N 33/53

(52) U.S. Cl. 800/298; 536/23.5; 435/320.1; 435/419; 530/350; 530/387.9; 435/468; 435/469; 435/470; 435/6; 435/7.1

(57) ABSTRACT

GmWRKY transcription factor genes and proteins from soybean. Plants that overexpress GmWRKY transcription factor genes and proteins to thereby increase stress tolerance. Methods for making such slants and methods for mimicking a stress tolerance phenotype using a GmWRKY modulator.

### RELATED APPLICATIONS

Priority is claimed to Chinese Application No. 200710176476.0, filed Oct. 29, 2007, which is incorporated herein by reference in its entirety.

### FIELD OF THE INVENTION

The present invention generally relates to compositions and methods for modulating plant characteristics such as plant stress tolerance.

### BACKGROUND OF THE INVENTION

One of the main objects of plant cultivation is the cultivation of a plant with increased stress tolerance. In higher plants, there are several pathways that mediate stress response. Several WRKY transcription factors have been successively cloned from different plants in response to environmental stresses, e.g., AtWRKYs in Arabidopsis thaliana, NtWRKY in tobacco, and StWRKY in potatoes. However, such abiotic stress related transcription factors from soybeans have not been cloned.

Soybean is an important oil producing plant and a main source of vegetable proteins, and its yield is severely affected by various environmental conditions. Accordingly, there is considerable practical significance in identifying mediators of stress tolerance in soybeans.

To meet this need, the present invention provides GmWRKY transcription factor genes and proteins from soybean. The present invention additionally provides plants that overexpress GmWRKY transcription factor genes and proteins to thereby increase stress tolerance, including increased tolerance to salinity and drought. Also provided are methods for making such plants and methods for mimicking a stress tolerance phenotype using a GmWRKY modulator.

### SUMMARY OF THE INVENTION

The present invention provides isolated WRKY transcription factor nucleic acids and proteins, vectors and cells expressing the disclosed nucleic acids, and antibodies that specifically bind to the disclosed protein. Representative WRKY transcription factor nucleic acids of the invention include nucleic acids comprising (a) a nucleotide sequence set forth as SEQ ID NO: 1; (b) a nucleic acid that specifically hybridizes to the complement of SEQ ID NO: 1 under stringent hybridization conditions; (c) a nucleotide sequence encoding a WRKY transcription factor protein comprising an amino acid sequence set forth as SEQ ID NO: 2; (d) a nucleotide sequence set forth as SEQ ID NO: 13; (e) a nucleic acid that specifically hybridizes to the complement of SEQ ID NO: 13 under stringent hybridization conditions; (f) a nucleotide sequence encoding a WRKY transcription factor protein comprising an amino acid sequence set forth as SEQ ID NO: 21; (g) a nucleotide sequence set forth as SEQ ID NO: 18; (h) a nucleic acid that specifically hybridizes to the complement of SEQ ID NO: 18 under stringent hybridization conditions; (i) a nucleotide sequence encoding a WRKY transcription factor protein comprising an amino acid sequence set forth as SEQ ID NO: 22; (j) a nucleotide sequence complementary to that of the nucleic acid of (a)-(i); and (k) a functional fragment of (a)-(j). Additionally, representative WRKY transcription factor nucleic acids of the invention include nucleic acids comprising (a) a nucleotide sequence that is at least 80% identical to SEQ ID NO: 1; (b) a nucleotide sequence that is at least 80% identical to SEQ ID NO: 13; (c) a nucleotide sequence that is at least 80% identical to SEQ ID NO: 18; (d) a nucleotide sequence encoding an WRKY transcription factor protein comprising an amino acid sequence that is at least 80% identical to SEQ ID NO: 2; (e) a nucleotide sequence encoding an WRKY transcription factor protein comprising an amino acid sequence that is at least 80% identical to SEQ ID NO: 21; and (f) a nucleotide sequence encoding an WRKY transcription factor protein comprising an amino acid sequence that is at least 80% identical to SEQ ID NO: 22. Representative WRKY transcription factor proteins of the invention include proteins comprising (a) an amino acid sequence at least 80% identical to SEQ ID NO: 2; (b) an amino acid sequence of SEQ ID NO: 2; (c) an amino acid sequence at least 80% identical to SEQ ID NO: 21; (d) an amino acid sequence of SEQ ID NO: 21; (e) an amino acid sequence at least 80% identical to SEQ ID NO: 22; (f) an amino acid sequence of SEQ ID NO: 22. Representative WRKY transcription factor proteins of the invention also include proteins which are related to stress tolerance of plants and derived from SEQ ID NO: 2, with one or more substitutions, deletions, and/or additions of the amino acid residues in SEQ ID NO: 2; SEQ ID NO: 21 and SEQ ID NO: 22. These substitutions, deletions, and/or additions of amino acid residues do not occur in a WRKYGQK domain which consists of a sequence of amino acid residues at positions 172 to 178 of SEQ ID NO: 2, positions 251-257 of SEQ ID NO: 21, or positions 117-123 of SEQ ID NO: 22, and do not occur in a zinc finger domain which consists of a sequence of amino acid residues at positions 192-197 of SEQ ID NO: 2 or at positions 136-141 of SEQ ID NO: 22. Representative nucleic acids of the invention also include nucleic acids encoding these representative proteins.

Also provided are plants expressing a heterologous WRKY transcription factor, including monocot and dicot plants and methods of producing such plants using the nucleotide sequences of the WRKY transcription factor genes disclosed herein. The plants expressing a WRKY transcription factor are characterized by increased expression of WRKY transcription factors, increased stress tolerance, and improved growth in normal or stress conditions.

Further provided are methods of identifying WRKY transcription factor binding agents and modulators. In one aspect of the invention, the method can comprise (a) providing a WRKY transcription factor protein; (b) contacting the WRKY transcription factor protein with one or more test agents or a control agent under conditions sufficient for binding; (c) assaying binding of a test agent to the isolated WRKY transcription factor protein; and (d) selecting a test agent that demonstrates specific binding to the WRKY transcription factor protein. In another aspect of the invention, the method can comprise (a) providing a cell recombinantly expressing a WRKY transcription factor protein; (b) contacting the cell with one or more test agents or a control agent; (c) assaying expression of the WRKY transcription factor gene; and (d) selecting a test agent that induces altered expression of the gene when contacted with the test agent as compared to the control agent. In another aspect of the invention, the method can comprise (a) providing a plant recombinantly expressing a WRKY transcription factor protein; (b) contacting the plant with one or more test agents or a control agent; (c) assaying survival of the plants under normal or abiotic stress growth conditions; and (d) selecting a test agent that promotes survival of the plants when in the presence of the test agent as compared to the control agent. Still further provided are methods of identifying modulators of a WRKY transcription factor in a plant comprising (a) providing a cell recombinantly expressing a WRKY transcription factor protein; (b) contacting the cell with one or more test agents or a control agent; (c) assaying expression of a WRKY transcription factor target gene; and (d) selecting a test agent that induces altered expression of the target gene, which gene is normally subject to WRKY transcription factor control, when contacted with the test agent as compared to the control agent.

Also provided are methods for conferring improved growth and/or stress tolerance upon a plant comprising introducing a WYRK nucleic acid disclosed herein into a plant cell to obtain a plant having improved growth and/or stress tolerance, for example, drought tolerance, salt tolerance, and/or low temperature tolerance.

### BRIEF DESCRIPTION OF THE DRAWINGS

FIG. 1 shows the expression levels of GmWRKY54 as determined by Northern hybridization in plants grown under conditions of salt stress (NaCl), drought stress, and low temperature stress (4° C.) for the indicated temporal durations. Expression of rRNA was used as a control.

FIG. 2 shows a schematic diagram of the structure of the pBin438-GmWRKY54 vector. NPT II, neomycin phosphotransferase II gene; 35S, CaMV 35S promoter; Ω, tobacco mosaic virus Ω sequence; GmWRKY54, GmWRKY54 open reading frame; NOS, terminator sequence.

FIG. 3 shows the expression level of GmWRKY54 and Arabidopsis thaliana tubulin (Attubulin) in control plants (Col-0) and GmWRKY54 transgenic plants 54-19 and 54-24 as determined by Northern hybridization.

FIG. 4A shows growth of control plants (Col-0) and GmWRKY54 transgenic plants 54-19 and 54-24 in (i) MS medium only, (ii) MS medium containing 150 mM, or (iii) MS medium containing 180 mM NaCl, and a further 8 days of growth under normal conditions.

FIG. 4B shows the survival rate of control plants (Col-0) and GmWRKY54 transgenic plants 54-19 and 54-24 in (i) MS medium only, (ii) MS medium containing 150 mM NaCl, or (iii) MS medium containing 180 mM NaCl, and a further 8 days of growth under normal conditions.

FIG. 5A shows plant growth of control plants (Col-0) and GmWRKY54 transgenic plants 54-19 and 54-24 under positive control conditions (CK) or at 26-28° C., 15%-20% of relative humidity, continuous illumination, and no watering for 18 days, followed by a further 3 days of watering (Drought).

FIG. 5B shows the survival rate of control plants (Col-0) and GmWRKY54 transgenic plants 54-19 and 54-24 under positive control conditions (CK) or at 26-28° C., 15%-20% of relative humidity, continuous illumination, and no watering for 18 days, followed by a further 3 days of watering.

FIG. 6A shows the expression level of putative GmWRKY54 target genes as determined by RT-PCR in wild type plants (Col-0) and GmWRKY54 transgenic plants 54-19 and 54-24 grown under normal conditions. Arabidopsis thaliana tubulin (Attubulin) expression was used as a control.

FIG. 6B shows the expression level of DREB2A, RD29B, and SZT as determined by Northern hybridization in wild type plants (Col-0) and GmWRKY54 transgenic plants 54-19 and 54-24 grown under normal conditions. 18S rRNA expression was used as a control.

FIG. 7 shows the expression levels of GmWRKY13 as determined by Northern hybridization in plants grown under conditions of salt stress (NaCl), drought stress, and low temperature stress (4° C.) for the indicated temporal durations (h, hours). Expression of rRNA was used as a control.

FIG. 8 shows the expression level of GmWRKY13 and Arabidopsis thaliana tubulin (Attubulin) in control plants (Col-0) and the GmWRKY13 transgenic plants 13-1 and 13-19 determined by Northern hybridization.

FIG. 9A shows the lateral root number in control plants (Col-0) and GmWRKY13 transgenic plants 13-1 and 13-19 following growth in MS medium only or MS medium containing 100 mM NaCl. Asterisks, significant difference in comparison with wild type plants (P<0.01).

FIG. 9B shows the lateral root length of control plants (Col-0) and GmWRKY13 transgenic plants 13-1 and 13-19 following growth in MS medium only or MS medium containing 100 mM NaCl.

FIG. 10 shows the expression level of putative GmWRKY13 target genes as determined by RT-PCR in wild type plants (Col-0) and GmWRKY13 transgenic plants 13-1 and 13-19 grown under normal conditions.

FIG. 11 shows the expression levels of GmWRKY21 as determined by Northern hybridization in plants grown under conditions of salt stress (NaCl), drought stress, and low temperature stress (4° C.) for the indications temporal durations (h, hours). Expression of rRNA was used as a control.

FIG. 12 shows the expression level of GmWRKY21 and Arabidopsis thaliana tubulin (Attubulin) in control plants (Col-0) and GmWRKY21 transgenic plants 21-6 and 21-11 as determined by Northern hybridization.

FIG. 13 shows the survival rate of control plants (Col-0) and GmWRKY21 transgenic plants 21-6 and 21-11 under positive control conditions (CK) or following exposure to −20° C. for 80 minutes with recovery at 22° C. for 16 days (Freezing).

### DETAILED DESCRIPTION

### I. WRKY Transcription Factor Nucleic Acids and Proteins

The present invention provides WRKY transcription factor nucleic acids and proteins, variants thereof, and modulators thereof. Previously described nucleic acids and proteins have not taught how to use such molecules for promoting stress tolerance in plants, as presently disclosed.

WRKY-type transcription factors are a large family of transcription factors. For example, in Arabidopsis thaliana, there are more than 100 members of the WRKY-type transcription factor family. Each of the members contains one or two WRKY domains which can bind to the W-box of promoters. The WRKY domain contains approximately 60 amino acids with an invariant WRKYGQK core sequence plus a novel zinc-finger motif. WRKY proteins are known to regulate the expression of target genes by specific binding to promoter regions containing the W-box sequence (T)TTGAC(C/T) (Eulgem et al., EMBO, 1999, 18:4689-99). The WRKY family is involved in various biological functions in plants, for example, resistance to pathogenic bacteria, death of plants, morphogenesis, and responses to abiotic stress. W-box, which is found in many gene promoters related to plant defense, mediates transcription induced by pathogen-derived stimuli. Infection of plants by viruses, bacteria, or fungi, or administration of molecular messengers, such as salicylic acid, can also induce synthesis of and increase the DNA binding activity of WRKY transcription factors. WRKY transcriptional activity regulates downstream genes to promote plant growth and plant tolerance to stressful growth conditions.

Representative WRKY transcription factor nucleic acids of the invention are set forth as SEQ ID NOs: 1, 13, and 18, which encode the representative WRKY transcription factor proteins of SEQ ID NOs: 2, 21, and 22. WRKY transcription factor variants and fragments encompassed by the present invention include variant proteins having WRKY transcription factor activity, e.g., that bind to the W-box sequence (T)TTGAC(C/T) or other upstream regulatory element to thereby regulate gene transcription, and that promote plant growth and stress tolerance.

I.A. WRKY Transcription Factor Nucleic Acids

Nucleic acids are deoxyribonucleotides or ribonucleotides and polymers thereof in single-stranded, double-stranded, or triplexed form. Unless specifically limited, the term encompasses nucleic acids containing known analogues of natural nucleotides that have similar properties as the reference natural nucleic acid. The terms nucleic acid molecule or nucleic acid may also be used in place of gene, cDNA, mRNA, or cRNA. Nucleic acids may be synthesized, or may be derived from any biological source, including any organism.

Representative nucleic acids of the invention comprise the nucleotide sequences of SEQ ID NOs: 1, 13 and 18, and substantially identical sequences encoding WRKY transcription factor proteins with substantially identical activity, for example, sequences at least 50% identical to SEQ ID NOs: 1, 13, or 18, such as at least 55% identical; or at least 60% identical; or at least 65% identical; such as at least 70% identical; or at least 75% identical; or at least 80% identical; or at least 85% identical; or at least 90% identical, or as at least 91% identical; or at least 92% identical; or at least 93% identical; or at least 94% identical; or at least 95% identical; or at least 96% identical; or at least 97% identical; or at least 98% identical; or at least 99% identical. Sequences are compared for maximum correspondence using a sequence comparison algorithm using the full-length sequence of SEQ ID NOs: 1, 13, or 18 as the query sequence, as described herein below, or by visual inspection.

Substantially identical sequences may be polymorphic sequences, i.e, alternative sequences or alleles in a population. An allelic difference may be as small as one base pair.

Substantially identical sequences may also comprise mutagenized sequences, including sequences comprising silent mutations. A mutation may comprise one or more residue changes, a deletion of one or more residues, or an insertion of one or more additional residues.

Substantially identical nucleic acids are also identified as nucleic acids that hybridize specifically to or hybridize substantially to the full length of SEQ ID NOs: 1, 13, or 18 under stringent conditions. In the context of nucleic acid hybridization, two nucleic acid sequences being compared may be designated a probe and a target. A probe is a reference nucleic acid molecule, and a target is a test nucleic acid molecule, often found within a heterogeneous population of nucleic acid molecules. A target sequence is synonymous with a test sequence.

A particular nucleotide sequence employed for hybridization studies or assays includes probe sequences that are complementary to at least an about 14 to 40 nucleotide sequence of a nucleic acid molecule of the present invention. Probes may comprise 14 to 20 nucleotides, or even longer where desired, such as 30, 40, 50, 60, 100, 200, 300, or 500 nucleotides or up to the full length of SEQ ID NOs: 1, 13, or 18. Such fragments may be readily prepared, for example by chemical synthesis of the fragment, by application of nucleic acid amplification technology, or by introducing selected sequences into recombinant vectors for recombinant production.

Specific hybridization refers to the binding, duplexing, or hybridizing of a molecule only to a particular nucleotide sequence under stringent conditions when that sequence is present in a complex nucleic acid mixture (e.g., total cellular DNA or RNA). Specific hybridization may accommodate mismatches between the probe and the target sequence depending on the stringency of the hybridization conditions.

Stringent hybridization conditions and stringent hybridization wash conditions in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen, Laboratory Techniques in Biochemistry and Molecular Biology-Hybridization with Nucleic Acid Probes, 1993, part I chapter 2, Elsevier, New York, N.Y. Generally, highly stringent hybridization and wash conditions are selected to be about 5° C. lower than the thermal melting point (Tm) for the specific sequence at a defined ionic strength and pH. Typically, under stringent conditions a probe will hybridize specifically to its target subsequence, but to no other sequences.

The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook et al., eds., Molecular Cloning: A Laboratory Manual, 1989, Cold Spring Harbor Laboratory Press, Cold Spring Harbor, N.Y., for a description of SSC buffer. Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4× to 6×SSC at 40° C. For short probes (e.g., about 10 to 50 nucleotides), stringent conditions typically involve salt concentrations of less than about 1M Na+ ion, typically about 0.01 to 1M Na+ ion concentration (or other salts) at pH 7.0-8.3, and the temperature is typically at least about 30° C. Stringent conditions may also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that may be used to identify nucleotide sequences that are substantially identical to reference nucleotide sequences of the present invention: a probe nucleotide sequence may hybridize to a target nucleotide sequence in 7% sodium dodecyl sulphate (SDS), 0.5M NaPO4, 1 mM EDTA at 50° C. followed by washing in 2×SSC, 0.1% SDS at 50° C.; for example, a probe and target sequence may hybridize in 7% sodium dodecyl sulphate (SDS), 0.5M NaPO4, 1 mM EDTA at 50° C. followed by washing in 1×SSC, 0.1% SDS at 50° C.; for example, a probe and target sequence may hybridize in 7% sodium dodecyl sulphate (SDS), 0.5M NaPO4, 1 mM EDTA at 50° C. followed by washing in 0.5×SSC, 0.1% SDS at 50° C.; for example, a probe and target sequence may hybridize in 7% sodium dodecyl sulphate (SDS), 0.5M NaPO4, 1 mM EDTA at 50° C. followed by washing in 0.1×SSC, 0.1% SDS at 50° C.; for example, a probe and target sequence may hybridize in 7% sodium dodecyl sulphate (SDS), 0.5M NaPO4, 1 mM EDTA at 50° C. followed by washing in 0.1×SSC, 0.1% SDS at 65° C.; for example, a probe and target sequence may hybridize in a solution of 6×SSC (0.5% SDS) at 65° C. followed by washing in 2×SSC (0.1% SDS) and 1×SSC (0.1% SDS) at 45° C.

A further indication that two nucleic acid sequences are substantially identical is that proteins encoded by the nucleic acids are substantially identical, share an overall three-dimensional structure, or are biologically functional equivalents. These terms are defined further herein below. Nucleic acid molecules that do not hybridize to each other under stringent conditions are still substantially identical if the corresponding proteins are substantially identical. This may occur, for example, when two nucleotide sequences comprise conservatively substituted variants as permitted by the genetic code.

The term conservatively substituted variants refers to nucleic acid sequences having degenerate codon substitutions wherein the third position of one or more selected (or all) codons is substituted with mixed-base and/or deoxyinosine residues. See Batzer et al., Nucleic Acids Res., 1991, 19:5081; Ohtsuka et al., J. Biol. Chem., 1985, 260:2605-2608; and Rossolini et al. Mol. Cell Probes, 1994, 8:91-98.

Nucleic acids of the invention also comprise nucleic acids complementary to SEQ ID NOs: 1, 13, and 18. Complementary sequences are two nucleotide sequences that comprise antiparallel nucleotide sequences capable of pairing with one another upon formation of hydrogen bonds between base pairs. As used herein, the term complementary sequences means nucleotide sequences which are substantially complementary, as may be assessed by the same nucleotide comparison methods set forth below, or is defined as being capable of hybridizing to the nucleic acid segment in question under relatively stringent conditions such as those described herein. Complementary nucleic acids include, for example, antisense oligonucleotides.

Nucleic acids of the invention also comprise nucleic acids of SEQ ID NOs: 1, 13, and 18, which have been altered for expression in organisms other than plants to account for differences in codon usage between plants and the other organism. For example, the specific codon usage in plants differs from the specific codon usage in certain microorganisms. Comparison of the usage of codons within a cloned microbial ORF to usage in plant genes (and in particular genes from the target plant) will enable an identification of the codons within the ORF that may be changed. Typically plant evolution has tended towards a strong preference of the nucleotides C and G in the third base position of monocotyledons, whereas dicotyledons often use the nucleotides A or T at this position. By modifying a gene to incorporate specific codon usage for a particular target transgenic species, many of the problems described below for GC/AT content and illegitimate splicing will be overcome.

Plant genes typically have a GC content of more than 35%. ORF sequences which are rich in A and T nucleotides can cause several problems in plants. Firstly, motifs of ATTTA are believed to cause destabilization of messages and are found at the 3′ end of many short-lived mRNAs. Secondly, the occurrence of polyadenylation signals such as AATAAA at inappropriate positions within the message is believed to cause premature truncation of transcription. In addition, monocotyledons may recognize AT-rich sequences as splice sites (see below).

The term subsequence refers to a sequence of nucleic acids that comprises a part of a longer nucleic acid sequence. An exemplary subsequence is a probe, described herein above, or a primer. The term primer as used herein refers to a contiguous sequence comprising about 8 or more deoxyribonucleotides or ribonucleotides, such as 10-20 nucleotides or 20-30 nucleotides of a selected nucleic acid molecule. The primers of the invention encompass oligonucleotides of sufficient length and appropriate sequence so as to provide initiation of polymerization on a nucleic acid molecule of the present invention.

Nucleic acids of the invention also comprise nucleic acids encoding a WRKY transcription factor protein set forth as SEQ ID NOs: 2, 21, or 22, or a WRKY transcription factor protein derived from SEQ ID NOs: 2, 21, or 22 containing one or more substitutions, deletions and/or additions of amino acid residues. Such nucleic acids encoding a WRKY transcription factor protein derived from SEQ ID NOs: 2, 21, or 22 may be obtained by deleting one or more codons from, making one or more missense mutations in, and/or linking one or more codons to the nucleic acid sequence of SEQ ID NOs: 1, 13, or 18, respectively. Variant WRKY nucleic acids of the invention include those nucleic acids encoding a functional WRKY transcription factor protein, as described herein below.

Representative nucleic acids of the invention also include nucleic acids consisting essentially of SEQ ID NOs: 1, 13, or 18, in as much as the nucleic acids are isolated from and do not contain additional nucleotide sequences with which the nucleic acids may be normally associated.

The invention also provides vectors comprising the disclosed nucleic acids, including vectors for recombinant expression, wherein a nucleic acid of the invention is operably linked to a functional promoter. When operably linked to a nucleic acid, a promoter is in functional combination with the nucleic acid such that the transcription of the nucleic acid is controlled and regulated by the promoter region. Vectors refer to nucleic acids capable of replication in a host cell, such as plasmids, cosmids, and viral vectors.

Nucleic acids of the present invention may be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions is also known in the art. See e.g., Sambrook et al. (eds.) Molecular Cloning: A Laboratory Manual, 1989, Cold Spring Harbor Laboratory Press, Cold Spring Harbor, N.Y.; Silhavy et al., Experiments with Gene Fusions, 1984, Cold Spring Harbor Laboratory Press, Cold Spring Harbor, N.Y.; Glover & Hames, DNA Cloning: A Practical Approach, 2nd ed., 1995, IRL Press at Oxford University Press, Oxford/N.Y.; Ausubel (ed.) Short Protocols in Molecular Biology, 3rd ed., 1995, Wiley, New York.

In another aspect of the invention, a method is provided for detecting a nucleic acid molecule that encodes a WRKY transcription factor protein. Such methods may be used to detect WRKY transcription factor gene variants or altered gene expression. Sequences detected by methods of the invention may be detected, subcloned, sequenced, and further evaluated by any measure well known in the art using any method usually applied to the detection of a specific DNA sequence. Thus, the nucleic acids of the present invention may be used to clone genes and genomic DNA comprising the disclosed sequences. Alternatively, the nucleic acids of the present invention may be used to clone genes and genomic DNA of related sequences. Levels of a WRKY transcription factor nucleic acid molecule may be measured, for example, using an RT-PCR assay. See Chiang, J. Chromatogr. A., 1998, 806:209-218, and references cited therein.

In another aspect of the invention, genetic assays using WRKY transcription factor nucleic acids may be performed for quantitative trait loci (QTL) analysis and to screen for genetic variants, for example by allele-specific oligonucleotide (ASO) probe analysis (Conner et al., Proc. Natl. Acad. Sci. USA, 1983, 80(1):278-282), oligonucleotide ligation assays (OLAs) (Nickerson et al., Proc. Natl. Acad. Sci. USA, 1990, 87(22):8923-8927), single-strand conformation polymorphism (SSCP) analysis (Orita et al., Proc. Natl. Acad. Sci. USA, 1989, 86(8):2766-2770), SSCP/heteroduplex analysis, enzyme mismatch cleavage, direct sequence analysis of amplified exons (Kestila et al., Mol. Cell, 1998, 1(4):575-582; Yuan et al., Hum. Mutat., 1999, 14(5):440-446), allele-specific hybridization (Stoneking et al., Am. J. Hum. Genet., 1991, 48(2):370-382), and restriction analysis of amplified genomic DNA containing the specific mutation. Automated methods may also be applied to large-scale characterization of single nucleotide polymorphisms (Wang et al., Am. J. Physiol., 1998, 274(4 Pt 2):H1132-1140; Brookes, Gene, 1999, 234(2):177-186). Preferred detection methods are non-electrophoretic, including, for example, the TAQMAN™ allelic discrimination assay, PCR-OLA, molecular beacons, padlock probes, and well fluorescence. See Landegren et al., Genome Res., 1998, 8:769-776 and references cited therein.

I.B. WRKY Transcription Factor Proteins

The present invention also provides isolated WRKY transcription factor polypeptides. Polypeptides and proteins each refer to a compound made up of a single chain of amino acids joined by peptide bonds. Representative WRKY transcription factor polypeptides are set forth as SEQ ID NOs: 2, 21 and 22. Additional polypeptides of the invention include WRKY transcription factor proteins with substantially identical activity, for example, sequences at least 50% identical to SEQ ID NOs: 2, 21 or 22, such as at least 55% identical; or at least 60% identical; or at least 65% identical; such as at least 70% identical; or at least 75% identical; or at least 80% identical; or at least 85% identical; or at least 90% identical, or as at least 91% identical; or at least 92% identical; or at least 93% identical; or at least 94% identical; or at least 95% identical; or at least 96% identical; or at least 97% identical; or at least 98% identical; or at least 99% identical. Sequences are compared for maximum correspondence using a sequence comparison algorithm using the full-length sequence of SEQ ID NOs: 2, 21, or 22 as the query sequence, as described herein below, or by visual inspection. Representative proteins of the invention also include polypeptides consisting essentially of SEQ ID NOs: 2, 21, or 22, in as much as the polypeptides are isolated from and do not contain additional amino acid sequences with which the polypeptides may be normally associated. The invention further encompasses polypeptides encoded by any one of the nucleic acids disclosed herein.

WRKY variant polypeptides include peptides with one or more deletions, additions, or substitutions that do not impact function of a WRKY transcription factor as described herein, including transcriptional regulatory activity, capacity to promote plant growth under normal conditions (see e.g., Example 12), or capacity to promote tolerance to stress growth conditions (see e.g., Examples 5-6 and 18). The GmWRKY54 polypeptide set forth in SEQ ID NO: 2 contains 323 amino acids, and it is a WRKY-type transcription factor in soybean. The WRKY domain containing the WRKY motif WRKYGQK is located at positions 172 to 178 from the N-terminus of SEQ ID NO: 2. A zinc finger domain is located at positions 192-197 from the N-terminus of SEQ ID NO: 2. Representative WRKY54 polypeptides of the invention may comprise substitutions, deletions and/or additions of amino acid residues, wherein the substitutions, deletions, and/or additions of the amino acid residues do not occur in the above domains of SEQ ID NO: 2. The GmWRKY13 polypeptide set forth in SEQ ID NO: 21 contains 324 amino acids, and it is a WRKY-type transcription factor in soybean. The WRKY domain containing the WRKY motif WRKYGQK is located at positions 251-257 of SEQ ID NO: 21. Representative WRKY13 polypeptides of the invention may comprise substitutions, deletions and/or additions of amino acid residues, wherein the substitutions, deletions, and/or additions of the amino acid residues does not occur in the above domain of SEQ ID NO: 21. Similarly, the GmWRKY21 polypeptide set forth in SEQ ID NO: 22 contains 196 amino acids, and it is a WRKY-type transcription factor in soybean. The WRKY domain containing the WRKY motif WRKYGKK is located at positions 117-123 of SEQ ID NO: 22. Representative WRKY21 polypeptides of the invention may comprise substitutions, deletions and/or additions of amino acid residues, wherein the substitutions, deletions, and/or additions of the amino acid residues does not occur in the above domain of SEQ ID NO: 22.

Polypeptides of the invention may comprise naturally occurring amino acids, synthetic amino acids, genetically encoded amino acids, non-genetically encoded amino acids, and combinations thereof. Polypeptides may include both L-form and D-form amino acids.

Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine.

The present invention also provides polypeptide variants that are functional fragments of a WRKY polypeptide, for example, fragments that have activity similar to that of a full-length WRKY protein. Functional polypeptide sequences that are longer than the disclosed sequences are also provided. For example, one or more amino acids may be added to the N-terminus or C-terminus of a polypeptide. Such additional amino acids may be employed in a variety of applications, including but not limited to purification applications. Methods of preparing elongated proteins are known in the art.

WRKY proteins of the invention include proteins comprising amino acids that are conservatively substituted variants of SEQ ID NOs: 2, 21, or 22. A conservatively substituted variant refers to a polypeptide comprising an amino acid in which one or more residues have been conservatively substituted with a functionally similar residue.

Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Isolated polypeptides of the invention may be purified and characterized using a variety of standard techniques that are known to the skilled artisan. See e.g., Schröder et al., The Peptides, 1965, Academic Press, New York; Bodanszky, Principles of Peptide Synthesis, 2nd rev. ed. 1993, Springer-Verlag, Berlin/N.Y.; Ausubel (ed.), Short Protocols in Molecular Biology, 3rd ed., 1995, Wiley, New York.

The present invention further provides methods for detecting a WRKY transcription factor polypeptide. The disclosed methods can be used, for example, to determine altered levels of WRKY transcription factor protein, for example, induced levels of WRKY transcription factor protein.

For example, the method may involve performing an immunochemical reaction with an antibody that specifically recognizes a WRKY transcription factor protein. Techniques for detecting such antibody-antigen conjugates or complexes are known in the art and include but are not limited to centrifugation, affinity chromatography and other immunochemical methods. See e.g., Ishikawa Ultrasensitive and Rapid Enzyme Immunoassay, 1999, Elsevier, Amsterdam/N.Y., United States of America; Law, Immunoassay: A Practical Guide, 1996, Taylor & Francis, London/Bristol, Pennsylvania, United States of America; Liddell et al., Antibody Technology, 1995, Bios Scientific Publishers, Oxford, United Kingdom; and references cited therein.

I.C. Nucleotide and Amino Acid Sequence Comparisons

The terms identical or percent identity in the context of two or more nucleotide or protein sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term substantially identical in regards to a nucleotide or protein sequence means that a particular sequence varies from the sequence of a naturally occurring sequence by one or more deletions, substitutions, or additions, the net effect of which is to retain biological function of a WRKY transcription factor nucleic acid or protein.

For comparison of two or more sequences, typically one sequence acts as a reference sequence to which one or more test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison may be conducted, for example, by the local homology algorithm of Smith & Waterman, Adv. Appl. Math, 1981, 2:482-489, by the homology alignment algorithm of Needleman & Wunsch, J. Mol. Biol., 1970, 48:443-453, by the search for similarity method of Pearson & Lipman, Proc. Natl. Acad. Sci. USA, 1988, 85:2444-2448, by computerized implementations of these algorithms (GAP, BESTFIT, FASTA, and TFASTA in the Wisconsin Genetics Software Package, Genetics Computer Group, Madison, Wis.), or by visual inspection. See generally, Ausubel (ed.), Short Protocols in Molecular Biology, 3rd ed., 1995, Wiley, New York.

A preferred algorithm for determining percent sequence identity and sequence similarity is the BLAST algorithm, which is described in Altschul et al., J. Mol. Biol., 1990, 215:403-410. Software for performing BLAST analyses is publicly available through the National Center for Biotechnology Information (http://www.ncbi.nlm.nih.gov/). The BLAST algorithm parameters determine the sensitivity and speed of the alignment. For comparison of two nucleotide sequences, the BLASTn default parameters are set at W=11 (wordlength) and E=10 (expectation), and also include use of a low-complexity filter to mask residues of the query sequence having low compositional complexity. For comparison of two amino acid sequences, the BLASTp program default parameters are set at W=3 (wordlength), E=10 (expectation), use of the BLOSUM62 scoring matrix, gap costs of existence=11 and extension=1, and use of a low-complexity filter to mask residues of the query sequence having low compositional complexity.

### II. System for Recombinant Expression of a WRKY Transcription Factor Protein

The present invention further provides a system for expression of a recombinant WRKY transcription factor protein. Such a system may be used for subsequent purification and/or characterization of a WRKY transcription factor protein. A system for recombinant expression of a WRKY transcription factor protein may also be used for identification of WRKY modulators, or targets of a WRKY transcription factor protein, as described further herein below. An expression system refers to a host cell comprising a heterologous nucleic acid and the protein encoded by the heterologous nucleic acid. For example, a heterologous expression system may comprise a host cell transfected with a construct comprising a WRKY transcription factor nucleic acid encoding a WRKY transcription factor protein operably linked to a promoter, or a cell line produced by introduction of WRKY transcription factor nucleic acids into a host cell genome. The expression system may further comprise one or more additional heterologous nucleic acids relevant to WRKY transcription factor function, such as targets of WRKY transcription factor activity. These additional nucleic acids may be expressed as a single construct or multiple constructs.

Isolated proteins and recombinantly produced proteins may be purified and characterized using a variety of standard techniques that are known to the skilled artisan. See e.g., Schröder et al., The Peptides, 1965, Academic Press, New York; Bodanszky, Principles of Peptide Synthesis, 2nd rev. ed. 1993, Springer-Verlag, Berlin/N.Y.; Ausubel (ed.), Short Protocols in Molecular Biology, 3rd ed., 1995, Wiley, New York. Additionally, recombinantly produced proteins may be purified by the addition of tags to the protein. Such tags may include Poly-Arg; Poly-His; FLAG; Strep-tag II; c-myc; or others, as known in the art.

II.A. Expression Constructs

A construct for expression of a WRKY transcription factor protein may include a vector sequence and a WRKY transcription factor nucleotide sequence, wherein the WRKY transcription factor nucleotide sequence is operably linked to a promoter sequence. A construct for recombinant WRKY transcription factor expression may also comprise transcription termination signals and sequences required for proper translation of the nucleotide sequence. Preparation of an expression construct, including addition of translation and termination signal sequences, is known to one skilled in the art.

The promoter may be any polynucleotide sequence which shows transcriptional activity in the chosen plant cells, plant parts, or plants. The promoter may be native or analogous, or foreign or heterologous, to the plant host and/or to the DNA sequence of the invention. Where the promoter is native or endogenous to the plant host, it is intended that the promoter is found in the native plant into which the promoter is introduced. Where the promoter is foreign or heterologous to the DNA sequence of the invention, the promoter is not the native or naturally occurring promoter for the operably linked DNA sequence of the invention. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley et al., Nucleic Acids Res., 1987, 15:2343-61. Also, the location of the promoter relative to the transcription start may be optimized. See e.g., Roberts et al., Proc. Natl. Acad. Sci. USA, 1979, 76:760-4. Many suitable promoters for use in plants are well known in the art.

For example, suitable constitutive promoters for use in plants include the promoters from plant viruses, such as the peanut chlorotic streak caulimovirus (PClSV) promoter (U.S. Pat. No. 5,850,019); the 35S promoter from cauliflower mosaic virus (CaMV) (Odell et al., Nature, 1985, 313:810-812); promoters of Chlorella virus methyltransferase genes (U.S. Pat. No. 5,563,328) and the full-length transcript promoter from figwort mosaic virus (FMV) (U.S. Pat. No. 5,378,619); the promoters from such genes as rice actin (McElroy et al. (1990) Plant Cell 2:163-171); ubiquitin (Christensen et al., Plant Mol. Biol., 1989, 12:619-632 and Christensen et al., Plant Mol. Biol., 1992, 18:675-689); pEMU (Last et al., Theor. Appl. Genet., 1991, 81:581-588); MAS (Velten et al., EMBO J., 1984, 3:2723-2730); maize H3 histone (Lepetit et al., Mol. Gen. Genet., 1992, 231:276-285 and Atanassova et al., Plant J., 1992, 2(3):291-300); Brassica napus ALS3 (PCT International Publication No. WO 97/41228); and promoters of various Agrobacterium genes (see U.S. Pat. Nos. 4,771,002; 5,102,796; 5,182,200; and 5,428,147).

Suitable inducible promoters for use in plants include the promoter from the ACE1 system which responds to copper (Mett et al., Proc. Natl. Acad. Sci. USA, 1993, 90:4567-4571); the promoter of the maize In2 gene which responds to benzenesulfonamide herbicide safeners (Hershey et al., Mol. Gen. Genetics, 1991, 227:229-237 and Gatz et al., Mol. Gen. Genetics, 1994, 243:32-38); and the promoter of the Tet repressor from Tn10 (Gatz et al., Mol. Gen. Genet., 1991, 227:229-237). Another inducible promoter for use in plants is one that responds to an inducing agent to which plants do not normally respond. An exemplary inducible promoter of this type is the inducible promoter from a steroid hormone gene, the transcriptional activity of which is induced by a glucocorticosteroid hormone (Schena et al., Proc. Natl. Acad. Sci. USA, 1991, 88:10421) or the recent application of a chimeric transcription activator, XVE, for use in an estrogen receptor-based inducible plant expression system activated by estradiol (Zuo et al., Plant J., 2000, 24:265-273). Other inducible promoters for use in plants are described in EP 332104, PCT International Publication Nos. WO 93/21334 and WO 97/06269. Promoters composed of portions of other promoters and partially or totally synthetic promoters can also be used. See e.g., Ni et al., Plant J., 1995, 7:661-676 and PCT International Publication No. WO 95/14098 describing such promoters for use in plants.

The promoter may include, or be modified to include, one or more enhancer elements to thereby provide for higher levels of transcription. Suitable enhancer elements for use in plants include the PClSV enhancer element (U.S. Pat. No. 5,850,019), the CaMV 35S enhancer element (U.S. Pat. Nos. 5,106,739 and 5,164,316) and the FMV enhancer element (Maiti et al., Transgenic Res., 1997, 6:143-156). See also PCT International Publication No. WO 96/23898.

Such constructs can contain a ‘signal sequence’ or ‘leader sequence’ to facilitate co-translational or post-translational transport of the peptide of interest to certain intracellular structures such as the chloroplast (or other plastid), endoplasmic reticulum, or Golgi apparatus, or to be secreted. For example, the construct can be engineered to contain a signal peptide to facilitate transfer of the peptide to the endoplasmic reticulum. A signal sequence is known or suspected to result in cotranslational or post-translational peptide transport across the cell membrane. In eukaryotes, this typically involves secretion into the Golgi apparatus, with some resulting glycosylation. A leader sequence refers to any sequence that, when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids including chloroplasts, mitochondria, and the like. Plant expression cassettes may also contain an intron, such that mRNA processing of the intron is required for expression.

Such constructs can also contain 5′ and 3′ untranslated regions. A 3′ untranslated region is a polynucleotide located downstream of a coding sequence. Polyadenylation signal sequences and other sequences encoding regulatory signals capable of affecting the addition of polyadenylic acid tracts to the 3′ end of the mRNA precursor are 3′ untranslated regions. A 5′ untranslated region is a polynucleotide located upstream of a coding sequence.

The termination region may be native with the transcriptional initiation region, may be native with the sequence of the present invention, or may be derived from another source. Convenient termination regions are available from the Ti-plasmid of A. tumefaciens, such as the octopine synthase and nopaline synthase termination regions, or the termination region of a plant gene, such as soybean storage protein. See also Guerineau et al., Mol. Gen. Genet., 1991, 262:141-144; Proudfoot, Cell, 1991, 64:671-674; Sanfacon et al., Genes Dev. 1991, 5:141-149; Mogen et al., Plant Cell, 1990, 2:1261-1272; Munroe et al., Gene, 1990, 91:151-158; Ballas et al., Nucleic Acids Res., 1989, 17:7891-7903; and Joshi et al., Nucleic Acid Res., 1987, 15:9627-9639.

Where appropriate, the vector and WRKY transcription factor sequences may be optimized for increased expression in the transformed host cell. That is, the sequences can be synthesized using host cell-preferred codons for improved expression, or may be synthesized using codons at a host-preferred codon usage frequency. Generally, the GC content of the polynucleotide will be increased. See e.g., Campbell et al., Plant Physiol., 1990, 92:1-11 for a discussion of host-preferred codon usage. Methods are known in the art for synthesizing host-preferred polynucleotides. See e.g., U.S. Pat. Nos. 6,320,100; 6,075,185; 5,380,831; and 5,436,391, U.S. Published Application Nos. 20040005600 and 20010003849, and Murray et al., Nucleic Acids Res., 1989, 17:477-498, herein incorporated by reference.

For example, polynucleotides of interest can be targeted to the chloroplast for expression. In this manner, where the polynucleotide of interest is not directly inserted into the chloroplast, the expression cassette may additionally contain a polynucleotide encoding a transit peptide to direct the nucleotide of interest to the chloroplasts. Such transit peptides are known in the art. See e.g., Von Heijne et al., Plant Mol. Biol. Rep., 1991, 9:104-126; Clark et al. J. Biol. Chem., 1989, 264:17544-17550; Della-Cioppa et al., Plant Physiol., 1987, 84:965-968; Romer et al., Biochem. Biophys. Res. Commun., 1993, 196:1414-1421; and Shah et al., Science, 1986, 233:478-481. The polynucleotides of interest to be targeted to the chloroplast may be optimized for expression in the chloroplast to account for differences in codon usage between the plant nucleus and this organelle. In this manner, the polynucleotides of interest may be synthesized using chloroplast-preferred codons. See e.g., U.S. Pat. No. 5,380,831, herein incorporated by reference.

A plant expression cassette (i.e., a WRKY transcription factor open reading frame operably linked to a promoter) can be inserted into a plant transformation vector, which allows for the transformation of DNA into a cell. Such a molecule may consist of one or more expression cassettes, and may be organized into more than one vector DNA molecule. For example, binary vectors are plant transformation vectors that utilize two non-contiguous DNA vectors to encode all requisite cis- and trans-acting functions for transformation of plant cells (Hellens et al., Trends in Plant Science, 2000, 5:446-451).

A plant transformation vector comprises one or more DNA vectors for achieving plant transformation. For example, it is a common practice in the art to utilize plant transformation vectors that comprise more than one contiguous DNA segment. These vectors are often referred to in the art as binary vectors. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a polynucleotide of interest (i.e., a polynucleotide engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker sequence and the sequence of interest are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as in understood in the art (Hellens et al., 2000). Several types of Agrobacterium strains (e.g., LBA4404, GV3101, EHA101, EHA105, etc.) can be used for plant transformation. The second plasmid vector is not necessary for introduction of polynucleotides into plants by other methods such as microprojection, microinjection, electroporation, polyethylene glycol, etc.

II.B. Host Cells

Host cells are cells into which a heterologous WRKY nucleic acid molecule of the invention may be introduced. Representative eukaryotic host cells include yeast and plant cells, as well as prokaryotic hosts such as E. coli and Bacillus subtilis. Preferred host cells for functional assays substantially or completely lack endogenous expression of a WRKY transcription factor protein.

A host cell strain may be chosen which effects the expression of the recombinant sequence, or modifies and processes the gene product in a specific manner. For example, different host cells have characteristic and specific mechanisms for the translational and post-translational processing and modification (e.g., glycosylation, phosphorylation of proteins). Appropriate cell lines or host cells may be chosen to ensure the desired modification and processing of the foreign protein expressed. For example, expression in a bacterial system may be used to produce a non-glycosylated core protein product, and expression in yeast will produce a glycosylated product.

The present invention further encompasses recombinant expression of a WRKY transcription factor protein in a stable cell line. Methods for generating a stable cell line following transformation of a heterologous construct into a host cell are known in the art. See e.g., Joyner, Gene Targeting: A Practical Approach, 1993, Oxford University Press, Oxford/N.Y. Thus, transformed cells, tissues, and plants are understood to encompass not only the end product of a transformation process, but also transgenic progeny or propagated forms thereof.

### III. Plants Expressing a Heterologous WRKY Transcription Factor Protein

The present invention also provides plants expressing a heterologous WRKY transcription factor, including those plants that express a WRKY transacription factor at elevated levels. The present invention also provides the generation of plants with conditional or inducible expression of a WRKY transcription factor protein.

Plants expressing a heterologous WRKY transcription factor may be monocot or dicot plants, for example, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers. Representative vegatables include tomatoes, lettuce, green beans, lima beans, peas, yams, onion, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. As used herein, a plant refers to a whole plant, a plant organ (e.g., leaves, stems, roots, etc.), a seed, a plant cell, a propagule, an embryo, and progeny of the same. Plant cells can be differentiated or undifferentiated (e.g., callus, suspension culture cells, protoplasts, leaf cells, root cells, phloem cells, pollen).

Plants expressing a heterologous WRKY transcription factor protein may be further modified at more than one WRKY transcription factor locus or at a locus other than a WRKY transcription factor locus to confer increased stress tolerance or other trait of interest. Representative desired traits include improved crop yield; increased seed yield; increased amino acid content; increased nitrate content; increased root number; increased tolerance to stress; insect resistance; tolerance to broad-spectrum herbicides; resistance to diseases caused by viruses, bacteria, fungi, and worms; and enhancement of mechanisms for protection from environmental stresses such as heat, cold, drought, and high salt concentration. Additional desired traits include output traits that benefit consumers, for example, nutritionally enhanced foods that contain more starch or protein, more vitamins, more anti-oxidants, and/or fewer trans-fatty acids; foods with improved taste, increased shelf-life, and better ripening characteristics; trees that make it possible to produce paper with less environmental damage; nicotine-free tobacco; ornamental flowers with new colors, fragrances, and increased longevity; etc. Still further, desirable traits that may be used in accordance with the invention include gene products produced in plants as a means for manufacturing, for example, therapeutic proteins for disease treatment and vaccination; textile fibers; biodegradable plastics; oils for use in paints, detergents, and lubricants; etc. For genetic modifications that confer traits associated with altered gene expression, or increased stress tolerance, the combination of transgenic expression of a heterologous WRKY transcription factor protein and a second genetic modification can produce a synergistic effect, i.e., a change in gene expression, or increased stress tolerance that is greater than the change elicited by either genetic modification alone.

For preparation of a plant expressing a heterologous WRKY transcription factor, introduction of a WRKY polynucleotide into plant cells is accomplished by one of several techniques known in the art, including but not limited to electroporation or chemical transformation (See e.g., Ausubel, ed. (1994) Current Protocols in Molecular Biology, John Wiley and Sons, Inc., Indianapolis, Ind. Markers conferring resistance to toxic substances are useful in identifying transformed cells (having taken up and expressed the test polynucleotide sequence) from non-transformed cells (those not containing or not expressing the test polynucleotide sequence). In one aspect of the invention, genes are useful as a marker to assess introduction of DNA into plant cells. Transgenic plants, transformed plants, or stably transformed plants, or cells, tissues or seed of any of the foregoing, refer to plants that have incorporated or integrated heterologous polynucleotides into the plant cell. Stable transformation refers to introduction of a polynucleotide construct into a plant such that it integrates into the genome of the plant and is capable of being inherited by progeny thereof.

In general, plant transformation methods involve transferring heterologous DNA into target plant cells (e.g., immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent (i.e., temperature and/or herbicide). The shoots are then transferred to a selective rooting medium for recovering rooted shoot or plantlet. The transgenic plantlet then grow into mature plant and produce fertile seeds (e.g., Hiei et al., Plant J., 1994, 6:271-282; Ishida et al., Nat. Biotechnol., 1996, 14:745-750). A general description of the techniques and methods for generating transgenic plants are found in Ayres et al., CRC Crit. Rev. Plant Sci., 1994, 13:219-239, and Bommineni et al., Maydica, 1997, 42:107-120. Since the transformed material contains many cells, both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants. Then molecular and biochemical methods can be used for confirming the presence of the integrated nucleotide(s) of interest in the genome of transgenic plant. For example, selectable markers, such as, enzymes leading to changes of colors or luminescent molecules (e.g., GUS and luciferase), antibiotic-resistant genes (e.g., gentamicin and kanamycin-resistance genes) and chemical-resistant genes (e.g., herbicide-resistance genes) may be used to confirm the integration of the nucleotide(s) of interest in the genome of transgenic plant. Alternatively, considering safety of the transgenic plants, the transformed plants can be selected under environmental stresses avoiding incorporation of any selectable marker genes.

Generation of transgenic plants may be performed by one of several methods, including but not limited to introduction of heterologous DNA by Agrobacterium into plant cells (Agrobacterium-mediated transformation), bombardment of plant cells with heterologous foreign DNA adhered to particles, and various other non-particle direct-mediated methods, including microinjection, electroporation, application of Ti plasmid, Ri plasmid, or plant virus vector and direct DNA transformation (e.g., Hiei et al., Plant J., 1994, 6:271-282; Ishida et al., Nat. Biotechnol., 1996, 14:745-750; Ayres et al., CRC Crit. Rev. Plant Sci., 1994, 13:219-239; Bommineni et al., Maydica, 1997, 42:107-120) to transfer DNA.

There are three common methods to transform plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (a) that the plant cells or tissues can be transformed by Agrobacterium and (b) that the transformed cells or tissues can be induced to regenerate into whole plants. The third method is transformation of seeds, apices or meristems with Agrobacterium. This method requires micropropagation.

The efficiency of transformation by Agrobacterium may be enhanced by using a number of methods known in the art. For example, the inclusion of a natural wound response molecule such as acetosyringone (AS) to the Agrobacterium culture has been shown to enhance transformation efficiency with Agrobacterium tumefaciens (Shahla et al., Plant Molec. Biol, 1987, 8:291-298). Alternatively, transformation efficiency may be enhanced by wounding the target tissue to be transformed. Wounding of plant tissue may be achieved, for example, by punching, maceration, bombardment with microprojectiles, etc. See e.g., Bidney et al., Plant Molec. Biol., 1992, 18:301-313.

In another aspect of the invention, the plant cells are transfected with vectors via particle bombardment (i.e., with a gene gun). Particle mediated gene transfer methods are known in the art, are commercially available, and include, but are not limited to, the gas driven gene delivery instrument described in U.S. Pat. No. 5,584,807, the entire contents of which are herein incorporated by reference. This method involves coating the polynucleotide sequence of interest onto heavy metal particles, and accelerating the coated particles under the pressure of compressed gas for delivery to the target tissue.

Other particle bombardment methods are also available for the introduction of heterologous polynucleotide sequences into plant cells. Generally, these methods involve depositing the polynucleotide sequence of interest upon the surface of small, dense particles of a material such as gold, platinum, or tungsten. The coated particles are themselves then coated onto either a rigid surface, such as a metal plate, or onto a carrier sheet made of a fragile material such as mylar. The coated sheet is then accelerated toward the target biological tissue. The use of the flat sheet generates a uniform spread of accelerated particles that maximizes the number of cells receiving particles under uniform conditions, resulting in the introduction of the polynucleotide sample into the target tissue.

Specific initiation signals may also be used to achieve more efficient translation of sequences encoding the polypeptide of interest. Such signals include the ATG initiation codon and adjacent sequences. In cases where sequences encoding the polypeptide of interest, its initiation codon, and upstream sequences are inserted into the appropriate expression vector, no additional transcriptional or translational control signals may be needed. However, in cases where only coding sequence, or a portion thereof, is inserted, heterologous translational control signals including the ATG initiation codon may be provided. Furthermore, the initiation codon should be in the correct reading frame to ensure translation of the entire insert. Heterologous translational elements and initiation codons may be of various origins, both natural and synthetic. The efficiency of expression may be enhanced by the inclusion of enhancers that are appropriate for the particular cell system that is used, such as those described in the literature (Scharf et al., Results Probl. Cell Differ., 1994, 20:125).

In another aspect of the present invention, at least one genomic copy corresponding to a nucleotide sequence of the present invention is modified in the genome of the plant by homologous recombination as further illustrated in Paszkowski et al., EMBO 1988, 7:4021-26. This technique uses the property of homologous sequences to recognize each other and to exchange nucleotide sequences between each by a process known in the art as homologous recombination. Homologous recombination can occur between the chromosomal copy of a nucleotide sequence in a cell and an incoming copy of the nucleotide sequence introduced in the cell by transformation. Specific modifications are thus accurately introduced in the chromosomal copy of the nucleotide sequence. In one aspect, the regulatory elements of the nucleotide sequences of the present invention are modified. Such regulatory elements are easily obtainable by screening a genomic library using the nucleotide sequences of the present invention, or a portion thereof, as a probe. The existing regulatory elements are replaced by different regulatory elements, thus altering expression of the nucleotide sequence.

The cells that have been transformed may be grown into plants in accordance with conventional ways. See e.g., McCormick et al., Plant Cell Rep., 1986, 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as transgenic seed) having a polynucleotide of the invention, for example, an expression cassette of the invention, stably incorporated into their genome.

Transgenic plants of the invention can be homozygous for the added WRKY polynucleotides, i.e., a transgenic plant that contains two added sequences, one sequence at the same locus on each chromosome of a chromosome pair. A homozygous transgenic plant can be obtained by sexually mating (selfing) an independent segregant transgenic plant that contains the added sequences according to the invention, germinating some of the seed produced and analyzing the resulting plants produced for enhanced enzyme activity (i.e., herbicide resistance) and/or increased plant yield relative to a control (native, non-transgenic) or an independent segregant transgenic plant.

It is to be understood that two different transgenic plants can also be mated to produce offspring that contain two independently segregating added, heterologous polynucleotides.

Selfing of appropriate progeny can produce plants that are homozygous for all added, heterologous polynucleotides that encode a polypeptide of the present invention. Back-crossing to a parental plant and outcrossing with a non-transgenic plant are also contemplated.

Following introduction of DNA into plant cells, the transformation or integration of the polynucleotide into the plant genome is confirmed by various methods such as analysis of polynucleotides, polypeptides and metabolites associated with the integrated sequence.

### IV. WRKY Transcription Factor Knockout Plants

The present invention also provides knockout plants comprising a disruption of a WRKY transcription factor locus to thereby achieve improved plant growth, reduced plant sensitivity to stress growth conditions, or increased tolerance to stress growth conditions. For example, GmWRKY13 transgenic plants show increased sensitivity to salt and osmotic stress (see Example 12), indicating that GmWRKY13 is a negative regulator of stress tolerance pathways and that inhibition of GmWRKY13 may be useful for reducing salt and osmotic stress sensitivity. A disrupted gene may result in expression of an altered level of full-length WRKY protein or expression of a mutated WRKY transcription factor protein.

WRKY transcription factor knockout plants as described herein may be monocots or dicots, including all plants identified herein above with respect to plants that overexpress a WRKY transcription factor gene. Plants with complete or partial functional inactivation of a WRKY transcription factor gene may be generated using standard techniques, such as T-DNA insertion as known in the art.

A knockout plant in accordance with the present invention may also be prepared using anti-sense, double-stranded RNA, or ribozyme WRKY transcription factor constructs, which are driven by a universal or tissue-specific promoter, to reduce levels of WRKY transcription factor gene expression in somatic cells, thus achieving a “knock-down” phenotype. The present invention also provides the generation of plants with conditional or inducible inactivation of a WRKY transcription factor.

The present invention also provides transgenic plants with specific “knocked-in” modifications in the disclosed WRKY transcription factor gene, for example, to create an over-expression mutant having a dominant negative phenotype. Thus, “knocked-in” modifications include the expression of both wild type and mutated forms of a nucleic acid encoding a WRKY transcription factor protein.

### V. WRKY Transcription Factor Protein Binding Partners and Modulators

The present invention further discloses assays to identify WRKY transcription factor protein binding partners and WRKY transcription factor modulators. WRKY transcription factor modulators are agents that alter chemical and biological activities or properties of a WRKY transcription factor protein. Modulation can comprise activation/induction or repression/inhibition. Such chemical and biological activities and properties may include, but are not limited to, WRKY transcription factor nucleic acid expression levels and expression levels of nucleic acids subject to WRKY transcription factor regulation. Methods of identifying activators/inducers involve assaying an enhanced level or quality of WRKY transcription factor function in the presence of one or more test agents. Conversely, methods of identifying repressors/inhibitors involve assaying a reduce level or quality of WRKY transcription factor function. Representative WRKY transcription factor modulators include small molecules as well as biological entities, as described herein below.

A control level or quality of WRKY transcription factor activity refers to a level or quality of wild type WRKY transcription factor activity, for example, when using a recombinant expression system comprising expression of SEQ ID NOs: 2, 21, or 22. When evaluating the activating/inducing or repressive/inhibitory capacity of a test agent, a control level or quality of WRKY transcription factor activity comprises a level or quality of activity in the absence of the test agent.

Significantly changed activity of a WRKY transcription factor protein refers to a quantifiable change in a measurable quality that is larger than the margin of error inherent in the measurement technique. For example, significant inhibition refers to WRKY transcription factor activity that is reduced by about 2-fold or greater relative to a control measurement, or an about 5-fold or greater reduction, or an about 10-fold or greater reduction. A significant enhancement refers to WRKY transcription factor activity that is increased by about 2-fold or greater relative to a control measurement, or an about 5-fold or greater increase, or an about 10-fold or greater increase.

An assay of WRKY transcription factor function may comprise determining a level of WRKY transcription factor gene expression; determining DNA binding activity of a recombinantly expressed WRKY transcription factor protein; determining an active conformation of a WRKY transcription factor protein; or determining activation of signaling events in response to binding of a WRKY transcription factor modulator (e.g., increased stress tolerance, inhibition of stress sensitivity, or improved plant growth). Activation of signaling events may include inhibition of a negative regulator of such signaling.

In accordance with the present invention there is also provided a rapid and high throughput screening method that relies on the methods described herein. This screening method comprises separately contacting a WRKY transcription factor protein with a plurality of test agents. In such a screening method the plurality of test agents may comprise more than about 10⁴ samples, or more than about 10⁵ samples, or more than about 10⁶ samples.

The in vitro and cellular assays of the invention may comprise soluble assays, or may further comprise a solid phase substrate for immobilizing one or more components of the assay. For example, a WRKY transcription factor protein, or a cell expressing a WRKY transcription factor protein, may be bound directly to a solid state component via a covalent or non-covalent linkage. Optionally, the binding may include a linker molecule or tag that mediates indirect binding of a WRKY transcription factor protein to a substrate.

V.A. Test Agents

A test agent refers to any agent that potentially interacts with a WRKY transcription factor nucleic acid or protein, including any synthetic, recombinant, or natural product. A test agent suspected to interact with a protein may be evaluated for such an interaction using the methods disclosed herein.

Representative test agents include but are not limited to peptides, proteins, nucleic acids, small molecules (e.g., organic and inorganic chemical compounds), antibodies or fragments thereof, nucleic acid-protein fusions, any other affinity agent, and combinations thereof. A test agent to be tested may be a purified molecule, a homogenous sample, or a mixture of molecules or compounds.

A small molecule refers to a compound, for example an organic compound, with a molecular weight of less than about 1,000 daltons, such as less than about 750 daltons, or less than about 600 daltons, or less than about 500 daltons. A small molecule may also have a computed log octanol-water partition coefficient in the range of about −4 to about +14, such as in the range of about −2 to about +7.5.

Representative nucleic acids that may be used to disrupt WRKY transcription factor function include antisense RNA and small interfering RNAs (siRNAs). See e.g., U.S. Published Application No. 20060095987. These inhibitory molecules may be prepared based upon a WRKY gene sequence and known features of inhibitory nucleic acids. See e.g., Van der Krol et al., Plant Cell, 1990, 2:291-299; Napoli et al., Plant Cell, 1990, 2:279-289; English et al., Plant Cell, 1996, 8:179-188; Waterhouse et al., Nature Rev. Genet., 2003, 4:29-38. As described herein below, disruption or inhibition of WRKY transcription factor function may be useful for inhibition of a negative regulator of a stress response to thereby reduce plant sensitivity to stress conditions.

Test agents may be obtained or prepared as a library or collection of molecules. A library may contain a few or a large number of different molecules, varying from about ten molecules to several billion molecules or more. A molecule may comprise a naturally occurring molecule, a recombinant molecule, or a synthetic molecule. A plurality of test agents in a library may be assayed simultaneously. Optionally, test agents derived from different libraries may be pooled for simultaneous evaluation.

Representative libraries include but are not limited to a peptide library (U.S. Pat. Nos. 6,156,511, 6,107,059, 5,922,545, and 5,223,409), an oligomer library (U.S. Pat. Nos. 5,650,489 and 5,858,670), an aptamer library (U.S. Pat. Nos. 7,338,762; 7,329,742; 6,949,379; 6,180,348; and 5,756,291), a small molecule library (U.S. Pat. Nos. 6,168,912 and 5,738,996), a library of antibodies or antibody fragments (U.S. Pat. Nos. 6,174,708, 6,057,098, 5,922,254, 5,840,479, 5,780,225, 5,702,892, and 5,667988), a library of nucleic acid-protein fusions (U.S. Pat. No. 6,214,553), and a library of any other affinity agent that may potentially bind to a WRKY transcription factor protein.

A library may comprise a random collection of molecules. Alternatively, a library may comprise a collection of molecules having a bias for a particular sequence, structure, or conformation, for example, as for inhibitory nucleic acids. See e.g., U.S. Pat. Nos. 5,264,563 and 5,824,483. Methods for preparing libraries containing diverse populations of various types of molecules are known in the art, for example as described in U.S. patents cited herein above. Numerous libraries are also commercially available.

V.B. Expression Assays

In one aspect of the invention, a modulator of WRKY transcription factor may be identified by assaying expression of a WRKY transcription factor nucleic acid. For example, a method of identifying a WRKY transcription factor activator useful for promoting stress tolerance may include the steps of (a) providing a cell recombinantly expressing a WRKY transcription factor protein, which normally functions as a positive regulator of a stress response; (b) contacting the cell with one or more test agents or a control agent; (c) assaying expression of a nucleic acid encoding the WRKY transcription factor protein; and (d) selecting a test agent that induces elevated expression of the nucleic acid when contacted with the test agent as compared to the control agent. As another example, a method of identifying a WRKY transcription factor repressor useful for promoting stress tolerance may include the steps of (a) providing a cell recombinantly expressing a WRKY transcription factor protein, which normally functions as a negative regulator of a stress response; (b) contacting the cell with one or more test agents or a control agent; (c) assaying expression of a nucleic acid encoding the WRKY transcription factor protein; and (d) selecting a test agent that induces reduced expression of the nucleic acid when contacted with the test agent as compared to the control agent.

V.C. Binding Assays

In another aspect of the invention, a method of identifying of a WRKY transcription factor modulator may include the steps of determining specific binding of a test agent to a WRKY transcription factor protein. For example, a method of identifying a WRKY transcription factor binding partner may comprise: (a) providing a WRKY transcription factor protein of SEQ ID NOs: 2, 21, or 22; (b) contacting the WRKY transcription factor protein with one or more test agents under conditions sufficient for binding; (c) assaying binding of a test agent to the isolated WRKY transcription factor protein; and (d) selecting a test agent that demonstrates specific binding to the WRKY transcription factor protein. Specific binding may also encompass a quality or state of mutual action such that binding of a test agent to a WRKY transcription factor protein is activating/inducing or repressive/inhibitory with respect to WRKY transcription factor function, as readily determined using functional assays as described herein.

Specific binding refers to a binding reaction which is determinative of the presence of the protein in a heterogeneous population of proteins and other biological materials. The binding of a test agent to a WRKY transcription factor protein may be considered specific if the binding affinity is about 1×10⁴M⁻¹ to about 1×10⁶M⁻¹ or greater. Specific binding also refers to saturable binding. To demonstrate saturable binding of a test agent to a WRKY transcription factor protein, Scatchard analysis may be carried out as described, for example, by Mak et al., J. Biol. Chem., 1989, 264:21613-21618.

Several techniques may be used to detect interactions between a WRKY transcription factor protein and a test agent without employing a known competitive inhibitor. Representative methods include, but are not limited to, Fluorescence Correlation Spectroscopy, Surface-Enhanced Laser Desorption/Ionization Time-Of-flight Spectroscopy, and BIACORE® technology, each technique described herein below. These methods are amenable to automated, high-throughput screening.

Fluorescence Correlation Spectroscopy (FCS) measures the average diffusion rate of a fluorescent molecule within a small sample volume. The sample size may be as low as 10³ fluorescent molecules and the sample volume as low as the cytoplasm of a single bacterium. The diffusion rate is a function of the mass of the molecule and decreases as the mass increases. FCS may therefore be applied to protein-ligand interaction analysis by measuring the change in mass and therefore in diffusion rate of a molecule upon binding. In a typical experiment, the target to be analyzed (e.g., a WRKY transcription factor protein) is expressed as a recombinant protein with a sequence tag, such as a poly-histidine sequence, inserted at the N-terminus or C-terminus. The expression is mediated in a host cell, such as E. coli, yeast, Xenopus oocytes, or mammalian cells. The protein is purified using chromatographic methods. For example, the poly-histidine tag may be used to bind the expressed protein to a metal chelate column such as Ni2+ chelated on iminodiacetic acid agarose. The protein is then labeled with a fluorescent tag such as carboxytetramethylrhodamine or BODIPY™ reagent (available from Molecular Probes of Eugene, Oreg.). The protein is then exposed in solution to the potential ligand, and its diffusion rate is determined by FCS using instrumentation available from Carl Zeiss, Inc. (Thornwood of New York, N.Y.). Ligand binding is determined by changes in the diffusion rate of the protein.

Surface-Enhanced Laser Desorption/Ionization (SELDI) was developed by Hutchens & Yip, Rapid Commun. Mass Spectrom., 1993, 7:576-580. When coupled to a time-of-flight mass spectrometer (TOF), SELDI provides a technique to rapidly analyze molecules retained on a chip. It may be applied to ligand-protein interaction analysis by covalently binding the target protein, or portion thereof, on the chip and analyzing by mass spectrometry the small molecules that bind to this protein (Worrall et al., Anal Chem., 1998, 70(4):750-756). In a typical experiment, a target protein (e.g., a GmWRKY54 protein) is recombinantly expressed and purified. The target protein is bound to a SELDI chip either by utilizing a poly-histidine tag or by other interaction such as ion exchange or hydrophobic interaction. A chip thus prepared is then exposed to the potential ligand via, for example, a delivery system able to pipet the ligands in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Ligands that specifically bind a target protein are identified by the stringency of the wash needed to elute them.

BIACORE® relies on changes in the refractive index at the surface layer upon binding of a ligand to a target protein (e.g., a WRKY transcription factor protein) immobilized on the layer. In this system, a collection of small ligands is injected sequentially in a 2-5 microliter cell, wherein the target protein is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein. In a typical experiment, a target protein is recombinantly expressed, purified, and bound to a BIACORE® chip. Binding may be facilitated by utilizing a poly-histidine tag or by other interaction such as ion exchange or hydrophobic interaction. A chip thus prepared is then exposed to one or more potential ligands via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics of on rate and off rate allows the discrimination between non-specific and specific interaction. See also Homola et al., Sensors and Actuators, 1999, 54:3-15 and references therein.

V.D. Conformational Assay

The present invention also provides methods of identifying WRKY transcription factor binding partners and modulators that rely on a conformational change of a WRKY transcription factor protein when bound by or otherwise interacting with a test agent. For example, application of circular dichroism to solutions of macromolecules reveals the conformational states of these macromolecules. The technique may distinguish random coil, alpha helix, and beta chain conformational states.

To identify binding partners and modulators of a WRKY transcription factor protein, circular dichroism analysis may be performed using a recombinantly expressed WRKY transcription factor protein. A WRKY transcription factor protein is purified, for example by ion exchange and size exclusion chromatography, and mixed with a test agent. The mixture is subjected to circular dichroism. The conformation of a WRKY transcription factor protein in the presence of a test agent is compared to a conformation of a WRKY transcription factor protein in the absence of the test agent. A change in conformational state of a WRKY transcription factor protein in the presence of a test agent identifies a WRKY transcription factor binding partner or modulator. Representative methods are described in U.S. Pat. Nos. 5,776,859 and 5,780,242. Activity of the binding partner or modulator may be assessed using functional assays, such assays include nitrate content, nitrate uptake, lateral root growth, seed yield, amino acid content or plant biomass, as described herein.

V.E. Functional Assays

In another aspect of the invention, a method of identifying a WRKY transcription factor modulator employs a functional WRKY transcription factor protein, for example, as set forth in SEQ ID NOs: 2, 21, or 22. Representative methods for identifying WRKY modulators include assaying WRKY transcription factor transcriptional regulatory activity (e.g., in a protoplast assay system) and determining a physiological change elicited by WRKY transcription factor activity, for example, increased stress tolerance (see e.g., Examples 5, 6, and 18) or inhibition of stress sensitivity.

For example, a method of identifying a WRKY transcription factor modulator may comprise (a) providing a cell expressing a WRKY transcription factor protein; (b) contacting the cell with one or more test agents or a control agent; (c) assaying expression of a WRKY transcription factor target gene; and (d) selecting a test agent that induces a change in expression of the target gene, which gene is normally subject to WRKY transcription factor control (e.g., DREB2A, ERD10, RD29B, ARF6, ABI1, and STZ), when contacted with the test agent as compared to the control agent. Modulators with both activating/inducing and repressive/inhibitory function may be useful in conferring stress tolerance, depending on the role of the WRKY transcription factor and the affected target genes. For example, plants with elevated expression of GmWRKY54 and GmWRKY21 show stress tolerance (see e.g., Examples 5, 6, and 18), indicating that activators/inducers of these transcription factors may provide a growth advantage under stress conditions. In contrast, stress sensitivity conferred by overexpression of GmWRKY13 indicates that it functions as a negative regulator for stress responses (see e.g., Example 12), such that reduced/repressed expression or activity of a GmWRKY13 transcription factor may be useful for optimal growth under stress conditions.

In accordance with the disclosed methods, cells expressing a WRKY transcription factor may be provided in the form of a kit useful for performing an assay of WRKY transcription factor function. For example, a test kit for detecting a WRKY transcription factor modulator may include cells transfected with DNA encoding a full-length WRKY transcription factor protein and a medium for growing the cells.

Assays of WRKY transcription factor activity that employ transiently transfected cells may include a marker that distinguishes transfected cells from non-transfected cells. A marker may be encoded by or otherwise associated with a construct for WRKY transcription factor expression, such that cells are simultaneously transfected with a nucleic acid molecule encoding WRKY transcription factor and the marker. Representative detectable molecules that are useful as markers include but are not limited to a heterologous nucleic acid, a protein encoded by a transfected construct (e.g., an enzyme or a fluorescent protein), a binding protein, and an antigen.

A method of identifying a WRKY transcription factor modulator may also comprise (a) providing a plant recombinantly expressing a WRKY transcription factor protein; (b) contacting the plant with one or more test agents or a control agent; (c) assaying survival of the plants under abiotic stress; and (d) selecting a test agent that promotes survival of the plants under abiotic stress when in the presence of the test agent as compared to the control agent.

Assays employing cells expressing a heterologous WRKY transcription factor or plants expressing a heterologous WRKY transcription factor may additionally employ control cells or plants that are substantially devoid of native WRKY transcription factor and proteins substantially similar to a WRKY transcription factor protein. When using transiently transfected cells, a control cell may comprise, for example, an untransfected host cell. When using a stable cell line expressing a WRKY transcription factor protein, a control cell may comprise, for example, a parent cell line used to derive the cell line expressing a WRKY transcription factor. When using plants, a control plant may express a heterologous WRKY transcription factor at elevated levels.

V.F. Rational Design

The knowledge of the structure of a native WRKY transcription factor protein provides an approach for rational design of WRKY transcription factor modulators. In brief, the structure of a WRKY transcription factor protein may be determined by X-ray crystallography and/or by computational algorithms that generate three-dimensional representations. See Saqi et al., Bioinformatics, 1999, 15:521-522; Huang et al., Pac. Symp. Biocomput, 2000, 230-241; and PCT International Publication No. WO 99/26966. Alternatively, a working model of an ENOD93 protein structure may be derived by homology modeling (Maalouf et al., J. Biomol. Struct. Dyn., 1998, 15(5):841-851). Computer models may further predict binding of a protein structure to various substrate molecules that may be synthesized and tested using the assays described herein above. Additional compound design techniques are described in U.S. Pat. Nos. 5,834,228 and 5,872,011.

A WRKY transcription factor protein is a soluble protein, which may be purified and concentrated for crystallization. A purified WRKY transcription factor protein may be crystallized under varying conditions of at least one of the following: pH, buffer type, buffer concentration, salt type, polymer type, polymer concentration, other precipitating ligands, and concentration of purified WRKY transcription factor. Methods for generating a crystalline protein are known in the art and may be reasonably adapted for determination of a WRKY transcription factor protein as disclosed herein. See e.g., Deisenhofer et al., J. Mol. Biol., 1984, 180:385-398; Weiss et al., FEBS Lett., 1990, 267:268-272; or the methods provided in a commercial kit, such as the CRYSTAL SCREEN™ kit (available from Hampton Research of Riverside, Calif., USA).

A crystallized GmWRKY54 protein may be tested for functional activity and differently sized and shaped crystals are further tested for suitability in X-ray diffraction. Generally, larger crystals provide better crystallography than smaller crystals, and thicker crystals provide better crystallography than thinner crystals. GmWRKY54 crystals may range in size from 0.1-1.5 mm. These crystals diffract X-rays to at least 10 Å resolution, such as 1.5-10.0 Å or any range of value therein, such as 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 or 3, with 3.5 Å or less being preferred for the highest resolution.

V.G. WRKY Transcription Factor Antibodies

In another aspect of the invention, a method is provided for producing an antibody that specifically binds a WRKY transcription factor protein. According to the method, a full-length recombinant WRKY transcription factor protein is formulated so that it may be used as an effective immunogen, and used to immunize an animal so as to generate an immune response in the animal. The immune response is characterized by the production of antibodies that may be collected from the blood serum of the animal.

An antibody is an immunoglobulin protein, or antibody fragments that comprise an antigen binding site (e.g., Fab, modified Fab, Fab′, F(ab′)₂ or Fv fragments, or a protein having at least one immunoglobulin light chain variable region or at least one immunoglobulin heavy chain region). Antibodies of the invention include diabodies, tetrameric antibodies, single chain antibodies, tretravalent antibodies, multispecific antibodies (e.g., bispecific antibodies), and domain-specific antibodies that recognize a particular epitope. Cell lines that produce anti-WRKY transcription factor antibodies are also encompassed by the invention.

Specific binding of an antibody to a WRKY transcription factor protein refers to preferential binding to a WRKY transcription factor protein in a heterogeneous sample comprising multiple different antigens. Substantially lacking binding describes binding of an antibody to a control protein or sample, i.e., a level of binding characterized as non-specific or background binding. The binding of an antibody to an antigen is specific if the binding affinity is at least about 10⁻⁷ M or higher, such as at least about 10⁻⁸ M or higher, including at least about 10⁻⁹M or higher, at least about 10⁻¹¹ M or higher, or at least about 10⁻¹² M or higher.

WRKY transcription factor antibodies prepared as disclosed herein may be used in methods known in the art relating to the expression and activity of WRKY transcription factor proteins, e.g., for cloning of nucleic acids encoding a WRKY transcription factor protein, immunopurification of a WRKY transcription factor protein, and detecting a WRKY transcription factor protein in a plant sample, and measuring levels of a WRKY transcription factor protein in plant samples. To perform such methods, an antibody of the present invention may further comprise a detectable label, including but not limited to a radioactive label, a fluorescent label, an epitope label, and a label that may be detected in vivo. Methods for selection of a label suitable for a particular detection technique, and methods for conjugating to or otherwise associating a detectable label with an antibody are known to one skilled in the art. WRKY antibodies of the invention may also be used as modulators (i.e., activators/inducers or repressors/inhibitors) of a WRKY transcription factor as useful to improve plant growth characteristics under normal or stress growth conditions.

### VI. Modulation of a WRKY Transcription Factor in Plants

The disclosed WRKY transcription factor binding partners and WRKY transcription factor modulators are useful both in vitro and in vivo for applications generally related to assessing responses to abiotic stress and for increasing stress tolerance in plants. In particular, activators/inducers of a WRKY transcription factor that is a positive regulator of a stress response (e.g., GmWRKY54 and GmWRKY21) may be used to increase plant stress tolerance, including increased tolerance to drought, cold, and high salt concentrations. Likewise, repressors of a WRKY transcription factor that is a negative regulator of stress response (e.g., GmWRKY13) may also be used to increase plant stress tolerance

The present invention provides that an effective amount of a WRKY transcription factor modulator is administered to a plant, i.e., an amount sufficient to elicit a desired biological response. For example, an effective amount of a WRKY transcription factor modulator may comprise an amount sufficient to elicit changed expression of a WRKY transcription factor or genes normally subject to WRKY transcription factor regulation (e.g., DRAB2A, ERD10, RD29B, ARF6, ABI1, and STZ). The directionality of the change depends on the role of the WRKY transcription factor or target genes in stress response (i.e., genes that normally function as positive regulators of a stress response are induced by a WRKY modulator of the invention, whereas genes that normally function as negative regulators of a stress response are inhibited by a WRKY modulator of the invention). An effective amount may also comprise an amount sufficient to confer increased tolerance to drought, cold, and high salt concentrations.

Plants that may benefit from WRKY transcription factor modulation include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassaya, coffee, coconut, pineapple, citrus trees, cocoa, tea, banana, avocado, fig, guava, mango, olive, papaya, cashew, macadamia, almond, oats, Arabidopsis thaliana, poplar, turfgrass, vegetables, ornamentals, and conifers. Representative vegetables include tomatoes, lettuce, green beans, lima beans, peas, yams, onions, and members of the genus Curcumis such as cucumber, cantaloupe, and musk melon. Representative ornamentals include, but are not limited to, azalea, hydrangea, hibiscus, roses, tulips, daffodils, petunias, carnation, poinsettia, and chrysanthemum. Any of the afore-mentioned plants may be wild type, inbred, or transgenic, e.g., plants strains and genetically modified plants as used in agricultural settings.

Plants treated with a WRKY transcription factor modulator may be transgenic, i.e., genetically modified at a WRKY transcription factor locus, or at a locus other than a WRKY transcription factor locus to confer increased stress tolerance or other trait of interest. Representative desired traits include improved crop yield; increased seed yield; increased amino acid content; increased nitrate content; increased tolerance to stress; insect resistance; tolerance to broad-spectrum herbicides; resistance to diseases caused by viruses, bacteria, fungi, and worms; and enhancement of mechanisms for protection from environmental stresses such as heat, cold, drought, and high salt concentration. Additional desired traits include output traits that benefit consumers, for example, nutritionally enhanced foods that contain more starch or protein, more vitamins, more anti-oxidants, and/or fewer trans-fatty acids; foods with improved taste, increased shelf-life, and better ripening characteristics; trees that make it possible to produce paper with less environmental damage; nicotine-free tobacco; ornamental flowers with new colors, fragrances, and increased longevity; etc. Still further, desirable traits that may be used in accordance with the invention include gene products produced in plants as a means for manufacturing, for example, therapeutic proteins for disease treatment and vaccination; textile fibers; biodegradable plastics; oils for use in paints, detergents, and lubricants; etc. For genetic modifications that confer traits associated with increased altered gene expression, or increased stress tolerance, the combination of treatment with a WRKY transcription factor modulator and a genetic modification can produce a synergistic effect, i.e., a change in gene expression, or increased stress tolerance that is greater than the change elicited by either genetic modification alone.

### EXAMPLES

The following examples have been included to illustrate modes of the invention. Certain aspects of the following examples are described in terms of techniques and procedures found or contemplated by the present co-inventors to work well in the practice of the invention. In light of the present disclosure and the general level of skill in the art, those of skill will appreciate that the following examples are intended to be exemplary only and that numerous changes, modifications, and alterations may be employed without departing from the scope of the invention.

### Example 1

### Cloning of GmWRKY54 cDNA

Soybean (Glycine max L.) cv. Kefeng No. 1 was used to isolate GmWRKY genes and to examine the expression patterns under various treatments. Total RNA was isolated essentially as described by Zhang et al., Theor. Appl. Genet., 1995, 91:361-366. Total RNA was treated with RNase-free DNase (Promega, Madison, Wis., USA) to remove genomic DNA contamination, and first-strand cDNA was synthesized using 4 μg of total RNA and a cDNA synthesis kit (Promega) in a reaction volume of 20 μL. A BLAST search in the Soybean EST database clustered 64 WRKY-like gene fragments which all contained the entire WRKY domain. Specific primer pairs were designed according to the WRKY transcription factor gene sequences for these 64 WRKY gene fragments after cloning and/or assembly of the unigenes (Tian et al., Theor. Appl. Genet., 2004, 108:903-913). The total volume of the PCR mixture was 20 μL, and contained 1 μL of first-strand cDNA, 0.5 mM of each primer, 1×PCR buffer, 0.4 mM deoxynucleoside triphosphate (dNTP) and 1 unit of Tag polymerase. The reaction was denatured at 94° C. for 3 minutes, followed by 32 cycles of 1 minute at 94° C., 1 minute at 54° C. and 1 minute at 2° C., with a final extension for 10 minutes at 72° C. A soybean tubulin gene, amplified with primers 5′-AACCTCCTCCTCATCGTACT-3′ (SEQ ID NO: 3) and 5′-GACAGCATCAGCCATTCA-3′ (SEQ ID NO: 4), was used as a control. The PCR products were separated on an agarose gel and quantified using an Imaging DensitoMeter (Bio-Rad, Hercules, Calif., USA).

Based on the sequence of the GmWRKY54 gene, a pair of primers was designed as follows:

(SEQ ID NO: 7)

5′-ATCAG AATTC ATGGA GAAGA AGGAG ATGGC-3′

(SEQ ID NO: 8)

5′-GATGC TGCAG CTACT CTTCT TTCAA CATGT-3′

The cDNA obtained by the reverse transcription was used as a template for PCR amplification using the primers of SEQ ID NOs:7 and 8. The product of the PCR amplification was determined by electrophoresis on a 0.8% agarose gel. The electrophoresis result showed a band of approximately 1 kb which was consistent with the expected value. This band was recovered by an agarose gel recovering kit (TIANGEN BIOTECH, Beijing, CHINA) and cloned into plasmid pGEM-T Easy (Promega). The recombinant vector was transformed into E. coli DH5α competent cells, as described in Cohen et al., Proc. Natl. Acad. Sci. USA, 1972, 69:2110). Cells containing the fragment recovered from the agarose gel were selected by using the ampicillin resistance gene and disruption of the β-galactosidase enzyme encoding gene on the pGEM-T Easy plasmid. A recombinant plasmid containing the fragment was subsequently isolated and the fragment was sequenced, primers complementary to the T7 and SP6 promoter sequences, which flank the multiple cloning site on the pGEM-T Easy plasmid. The isolated fragment, designated GmWRKY54, contained 972 deoxyribonucleotides. The open reading frame of GmWRKY54 has a nucleotide sequence set forth as SEQ ID NO: 1 and encodes a polypeptide with an amino acid sequence set forth as SEQ ID NO: 2. The recombinant vector containing the GmWRKY54 gene of SEQ ID NO: 1 was designated as pTE-GmWRKY54.

### Example 2

### Expression of GmWRKY4 Under Environmental Stress Conditions

Seeds of soybean (Glycine max L.) cv. Kefeng No. 1 were germinated in pots containing vermiculite, and 2 week old seedlings were used in the following treatments. For NaCl treatment, the roots of the seedlings were immersed in solutions containing 200 mM NaCl for the indicated times. For dehydration treatment, the plants were carefully pulled out, transferred on to filter paper and allowed to dry for the indicated times. Low-temperature treatment was conducted by transferring seedlings to a beaker containing water precooled to 4° C., and maintaining the seedlings at 4° C. Leaves from all the treatments above were harvested at 0, 0.5, 1, 3, 6, and 12 hours and stored at −70° C. for RNA isolation. 1 g of the fresh leaves were ground down in liquid nitrogen and suspended in 4 mol/L guanidinium thiocyanate. The resulting mixture was extracted by acidic phenol and chloroform, and absolute alcohol was added into the supernatant to precipitate the total RNA.

For RNA gel blot analysis, 20 μg of total RNA for each lane was denatured, fractionated on a 1.2% agarose gel containing formaldehyde, and blotted on to a a HYBOND® N+ nylon membrane in 20× standard saline citrate (SSC). Northern hybridization was carried out at 65° C. using [α-32P]-dCTP-labeled GmWRKY cDNA as a probe. After hybridization, membranes were washed in 2×SSC plus 0.1% sodium dodecylsulphate (SDS) at 45° C. for 15 minutes and in 1×SSC plus 0.1% SDS at 45° C. for 5 minutes. The membranes were then autoradiographed using a phosphoimaging system (Amersham Pharmacia, Piscataway, N.J., USA). The following primers were used for GmWRKY54 probe template synthesis:

| | |
|---|---|
| 5′-ATGGAGAAGAAGGAGATGGC-3′ | (SEQ ID NO: 5) |
| 5′-CTACTCTTCTTTCAACATGT-3′. | (SEQ ID NO: 6) |

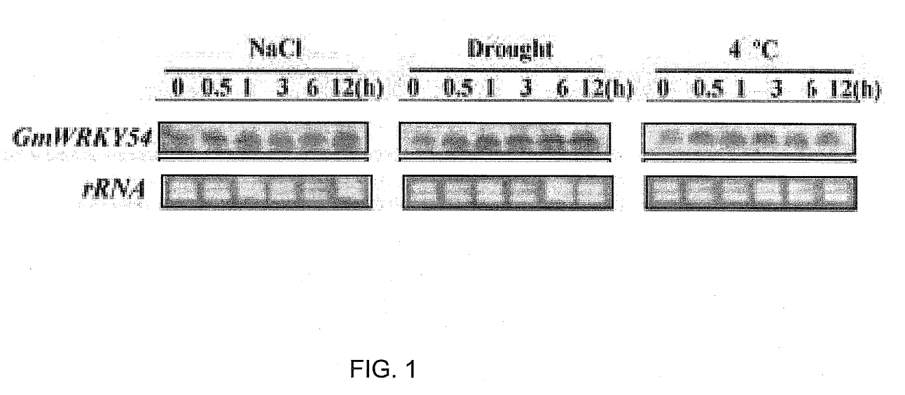

As shown in FIG. 1, the expression of GmWRKY54 was significantly induced by the stress of salt, drought, or low temperature of 4° C.

### Example 3

### Construction of a GmWRKY4 Expression Vector pBin438-GmWRKY54

The cDNA obtained by reverse transcription of total RNA from (Glycine max L.) cv. Kefeng No. 1 was PCR amplified with the following primers specific for GmWRKY54 (SEQ ID NO: 1) and also containing BamHI and KpnI linker sequences:

(SEQ ID NO: 9)

5′-ATC AGG ATC CAT GAC AGT AGA TCT GGT AGG-3′

(SEQ ID NO: 10)

5′-GAT GGG TAC CTG ACA GTA GAT CTG GTA GGT G-3′.

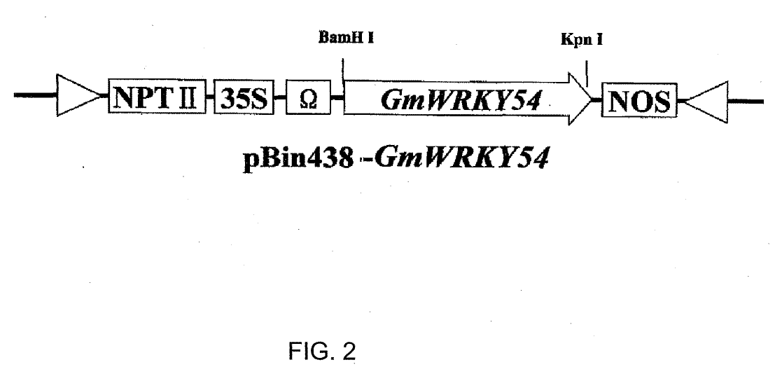

The amplified product was digested with BamHI and KpnI, recovered, and inserted between the BamHI and KpnI restriction sites in the sense orientation downstream of the CaMV 35S promoter in the plant binary expression vector pBin438 (Tai-yuan Li, et al., J Science in China (Column B), 1994, 24(3): 276-282). The resulting vector was designated as pBin438-GmWRKY54 (FIG. 2).

### Example 4

### Preparation of GmWRKY54 Transgenic Plants

Agrobacterium tumefaciens AGL1 was transformed with the pBin438-GmWRKY54 vector by electroporation. PCR was used to confirm transformation of the Agrobacterium tumefaciens AGL1 with the pBin438-GmWRKY54 vector. Agrobacterium tumefaciens AGL1 containing the pBin438-GmWRKY54 vector was transformed into wild type Arabidopsis thaliana (Columbia ecotype) (Col-0) by the floral dip method (Clough et al., Plant Journal, 1998, 16:6, 735-743). Selection of transgenic plants was performed on Murashige and Skoog (MS) selection media containing kanamycin (50 mg/L) to obtain the plants of T₀ generation. At the 4-6 leaf stage, plants of T₀ generation were transferred onto vermiculite. Ten control plants of T₀ generation were prepared as above using an empty pBin438 vector. PCR was performed to identify GmWRKY54 transgenic plants of the T₀ generation, control plants of T₀ generation transformed by blank vector, and wild type plants using the following primers:

(SEQ ID NO: 9)

5′-ATC AGG ATC CAT GAC AGT AGA TCT GGT AGG-3′

(SEQ ID NO: 10)

5′-GAT GGG TAC CTG ACA GTA GAT CTG GTA GGT G-3′.

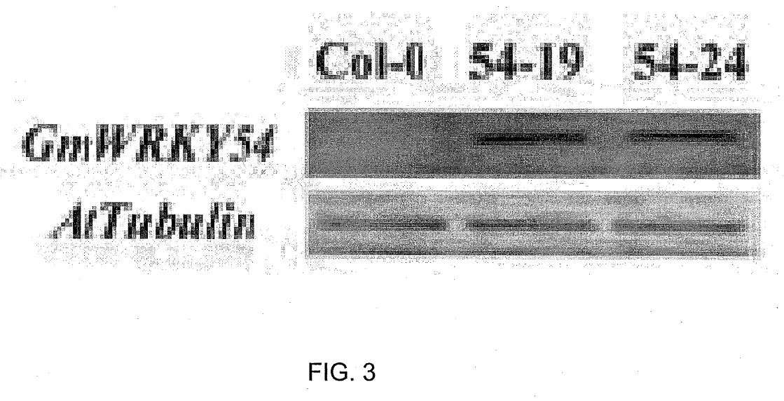

The GmWRKY54 gene was detected in 12 of 25 GmWRKY54 transgenic plants of T₀ generation, but in none of 10 wild type plants and 10 control plants transformed by blank vector. The positive plants of T₀ generation were selfed to obtain seeds, which were planted individually and selected on MS selection media containing kanamycin (50 mg/L) to obtain the plants of T₁ generation. This process of selfing and selection was repeated until a T₃ generation was obtained. In total 12 plants of the T₃ generation, which are hereditarily stable, were obtained. Expression of GmWRKY54 was verified by Northern hybridization using the GmWRKY54 gene (SEQ ID NO: 1) as the probe, tubulin expression was used as an internal control. GmWRKY54 was highly expressed in two GmWRKY54 transgenic plants of T₃ generation (54-19 and 54-24) and was expressed at an undetectable level in control plants transformed by blank vector (FIG. 3).

### Example 5

### Salt Stress Tolerance of GmWRKY54 Transgenic Plants

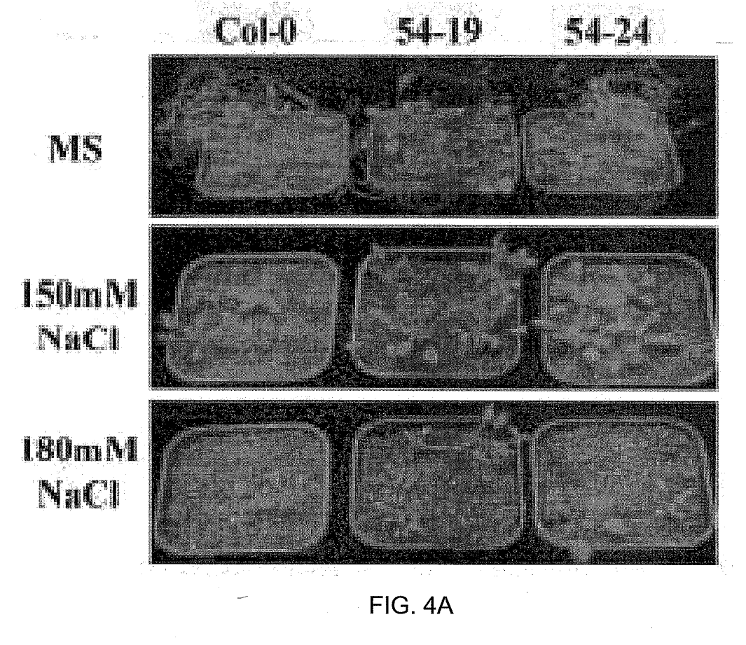

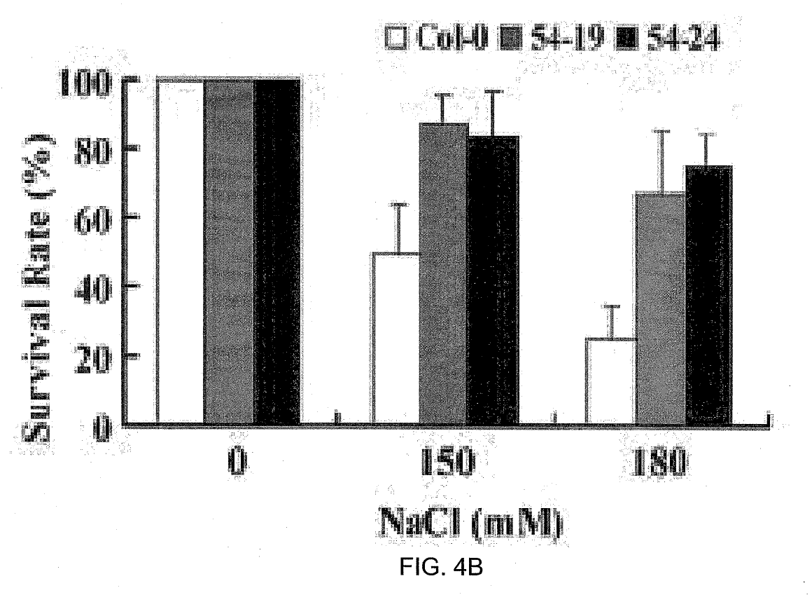

Two week old seedlings of the GmWRKY54 transgenic plant strains 54-19 and 54-24 of the T3 generation and control plants transformed by blank vector were grown in MS medium only or MS medium containing 150 mM or 180 mM NaCl. After 16 days, no obvious difference was observed at 150 mM NaCl, however, plants treated with 180 mM NaCl should more severe stress symptoms as compared to GmWRKY54 transgenic plants. The seedlings were then transferred to soil and grown for a further 8 days under normal conditions. The experiment was performed in triplicate with 15 plants for each strain. Plant growth at the end of the 8 day recovery period was observed and recorded (FIG. 4A). Survival rates of the plants was also determined (FIG. 4B).

GmWRKY54 transgenic plants 54-19 and 54-24 showed improved growth in salt stress conditions as compared to control plants (FIG. 4A). The survival rate of control plants (Col-0) transformed by blank vector and grown in MS medium containing 150 mM NaCl was 50%±11% (FIG. 4B). The survival rate of GmWRKY54 transgenic plants of 54-19 strain, grown under the same conditions, was 85%±5% (FIG. 4B). Likewise, the survival rate of GmWRKY54 transgenic plants of 54-24 strain, grown under the same conditions was 81%±7% (FIG. 4B). Following growth in MS medium containing 150 mM NaCl and subsequent growth under normal conditions, the survival rate of control plants (Col-0) transformed by blank vector was only 25%±5% (FIG. 4B). The mean survival rate of GmWRKY54 transgenic plants 54-19 and 54-24 was 70%±14% (FIG. 4B).

### Example 6

### Drought Stress Tolerance of GmWRKY54 Transgenic Plants

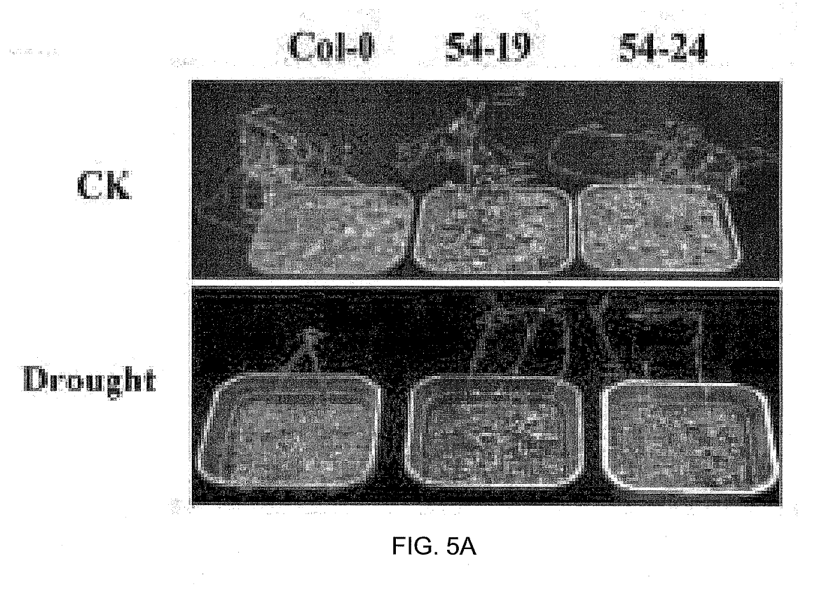

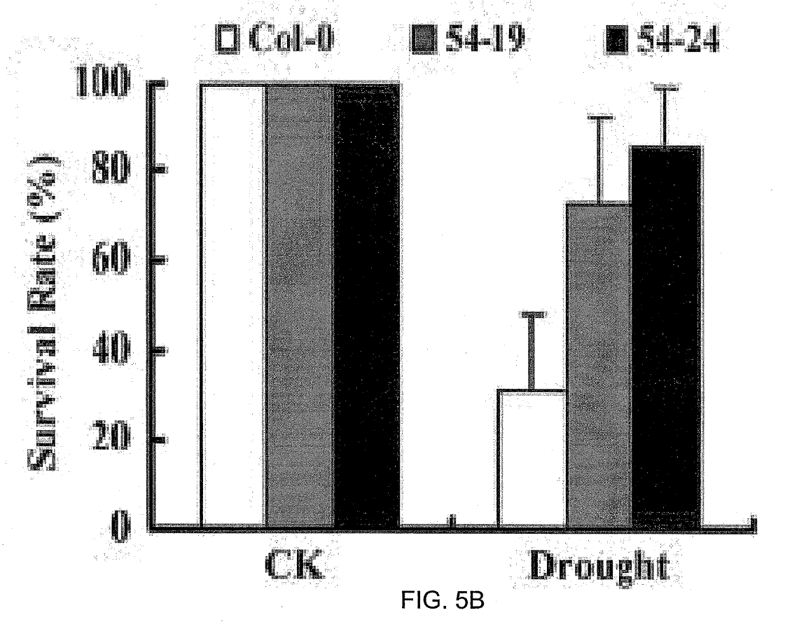

Three week old seedlings of the GmWRKY54 transgenic plant strains 54-19 and 54-24 of the T3 generation and control plants transformed by blank vector were grown under the following conditions: 26-28° C., 15%-20% of relative humidity, continuous illumination, and no watering for 18 days. Following this, the plants were watered for 3 days and plant growth at the end of the 3 days was observed and recorded (FIG. 5A). Survival rates of the plants was also determined (FIG. 5B). The experiment was performed in triplicate with 15 plants for each strain.

GmWRKY54 transgenic plants 54-19 and 54-24 showed improved growth in drought conditions as compared to control plants (FIG. 5A). Under normal conditions, the survival rate of all plants was 100%, but under the drought stress, the survival rate of control plants (Col-0) transformed by blank vector was 31.3%±14%, the survival rate of 54-19 plant strain was 72.9%±15%, and the survival rate of 54-24 plant strain was 85.4%±8% (FIG. 5B).

### Example 7

### Expression GmWRKY54 Target Genes

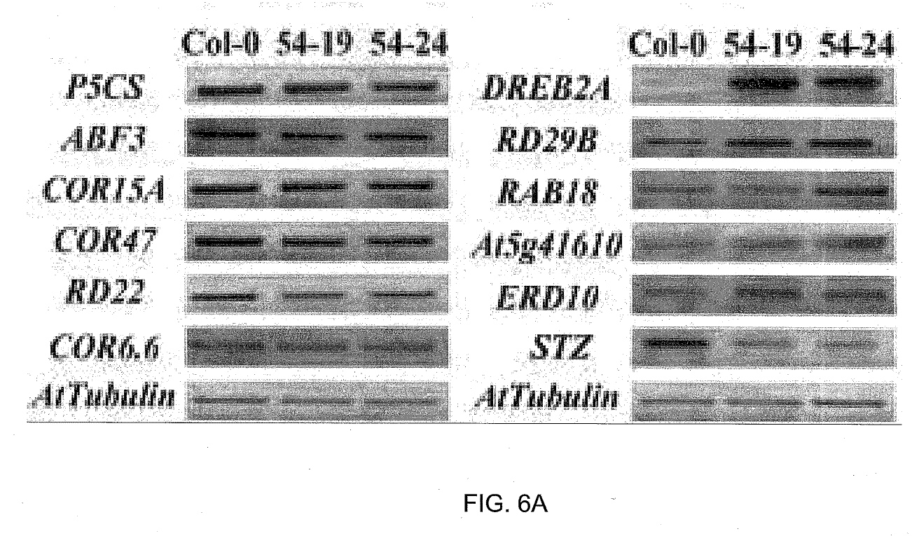

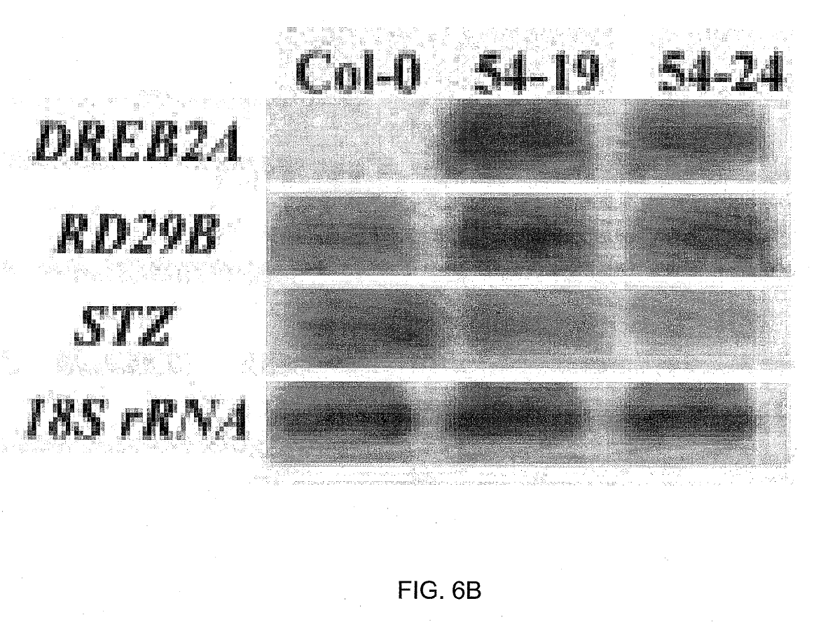

RNA samples were isolated from untreated 2-week-old seedlings of wild type plants (Cot-0) and GmWRKY54-transgenic lines (54-19, 54-24). A number of genes that contain four or more WRKY domain-binding sites in their promoter regions, together with several stress-related genes, were selected as candidate target genes for RT-PCR analysis (Liu et al., Plant Cell, 1998, 10: 1391-1406). The tubulin gene was used as an internal control. The expression of DREB2A, —RD29B, and ERD10 was up-regulated in GmWRKY54 transgenic plants, whereas STZ expression was down-regulated (FIG. 6A). Northern hybridization analysis was also performed using 18S rRNA as a loading control to confirm that the expression level of three of these genes (DRE132A, RD29B, and STZ) was different in GmWRKY transgenic plants compared to wild type plants (FIG. 6B).

### Example 8

### Cloning of GmWRKY13 cDNA

A GmWRKY13 cDNA was cloned using RT-PCR essentially as described for GmWRKY54 in Example 1. The following primers were used for GmWRKY13 probe template synthesis:

| | |
|---|---|
| 5′-ATGACAGTAGATCTGGTAGG-3′ | (SEQ ID NO: 11) |
| 5′-TTAAACAGGATGAGCGGCAA-3′. | (SEQ ID NO: 12) |

The recovered open reading frame of GmWRKY13 has a nucleotide sequence set forth as SEQ ID NO: 13 and encodes a polypeptide with an amino acid sequence set forth as SEQ ID NO: 21.

### Example 9

### Expression of GmWRKY13 Under Environmental Stress Conditions

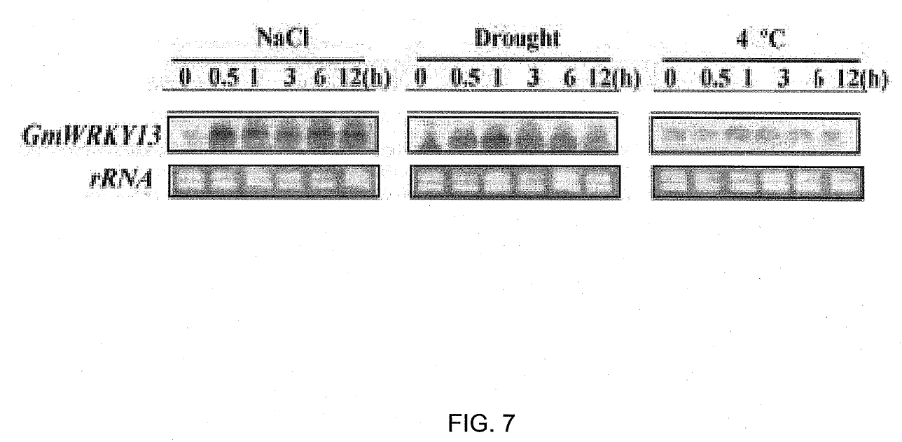

GmWRKY13 expression under environmental stress conditions was assessed essentially as described for GmWRKY54 in Example 2. Total RNA was analyzed by Northern hybridization using GmWRKY13 DNA as a probe. As shown in FIG. 7, the expression of GmWRKY13 was significantly induced by the stress of salt, drought, or low temperature of 4° C.

### Example 10

### Construction of a GmWRKY13 Expression Vector pBin438-GmWRKY13

The cDNA obtained by reverse transcription of total RNA from (Glycine max L.) cv. Kefeng No. 1 was PCR amplified with the following primers specific for GmWRKY13 (SEQ ID NO: 13) and also containing BamHI and KpnI linker sequences:

(SEQ ID NO: 14)

5′-ATCAGGATCCATGACAGTAGATCTGGTAGG-3′

(SEQ ID NO: 15)

5′-GATGGGTACCTGACAGTAGATCTGGTAGGTG-3′.

The amplified product was digested with BamHI and KpnI, recovered, and inserted between the BamHI and KpnI restriction sites in the sense orientation downstream of the CaMV ³⁵S promoter in the plant binary expression vector pBin438 (Tai-yuan Li, et al., J. Science in China (Column B), 1994, 24(3): 276-282). The resulting vector was designated as pBin438-GmWRKY13.

### Example 11

### Preparation of GmWRKY13 Transgenic Plants

Arabidopsis thaliana were transformed using pBin438-GmWRKY13 essentially as described in Example 4. PCR was performed to identify GmWRKY13 transgenic plants of the T₀ generation using the following primers:

(SEQ ID NO: 14)

5′-ATCAGGATCCATGACAGTAGATCTGGTAGG-3′

(SEQ ID NO: 15)

5′-GATGGGTACCTGACAGTAGATCTGGTAGGTG-3′.

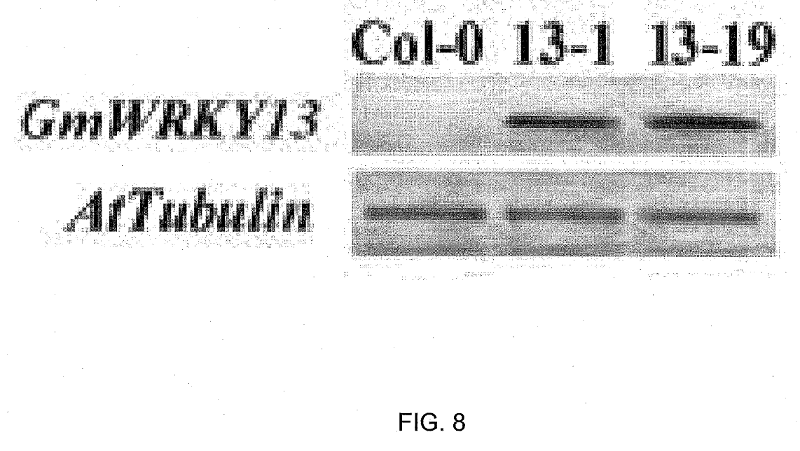

Positive plants of the T₀ generation were subjected to repeated selfing and selection until a T₃ generation was obtained. Expression of GmWRKY13 was verified by Northern hybridization using the GmWRKY13 gene (SEQ ID NO: 13) as a probe. Tubulin expression was used as an internal control. The GmWRKY13 gene was highly expressed in two GmWRKY13 transgenic plants of T₃ generation (13-1 and 13-19), and was expressed at an undetectable level in control plants transformed by blank vector (FIG. 8).

### Example 12

### Salt Stress and Osmotic Stress Sensitivity of GmWRKY13 Transgenic Plants

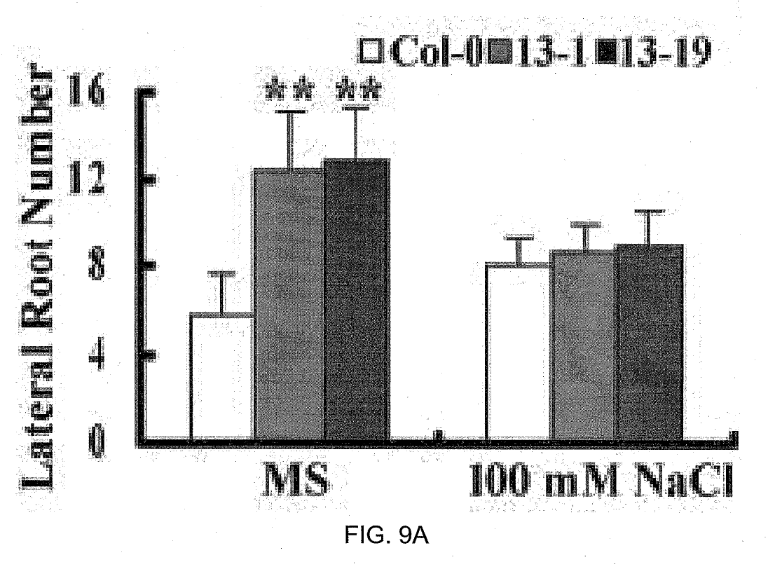

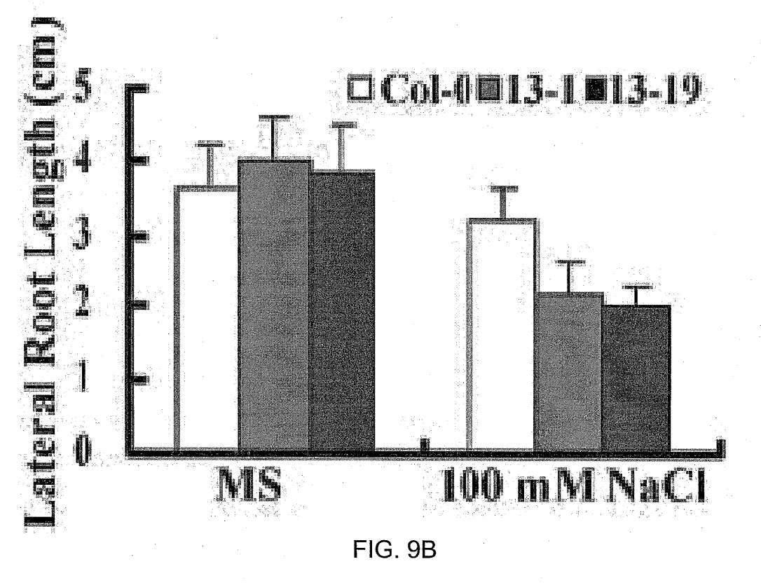

Five day old seedlings of GmWRKY13 transgenic plant strains 13-1 and 13-19 of the T₃ generation and control plants transformed by blank vector were grown in MS medium only or MS medium containing 100 mM. After two weeks, the numbers of lateral roots (≧5 mm) and the lateral root length from 20 plants were measured. The experiment was performed in triplicate with 20 plants for each strain. Lateral root number is shown in FIG. 9A and lateral root length is shown in FIG. 9B.

Under normal growth conditions in MS, GmWRKY13 transgenic plants produced more lateral roots than the wild type plants: average number of lateral roots per seedling was 5.7 for wild type plants, and 12.4 and 12.7 for transgenic lines 13-1 and 13-19, respectively (FIG. 9A). These results indicate that expression of GmWRKY13 may confer a growth advantage under favorable growth conditions. The lateral root length was not significantly affected in the transgenic lines and wild type plants under normal conditions (FIG. 9B). Under salt stress conditions, the lateral root number was not significantly different between the transgenic lines and wild type plants (FIG. 9B). However, the lateral root length was reduced in GmWRKY13 transgenic plants in comparison with the wild type plants on salt treatment (FIG. 9B).

To assess osmotic stress responses, seven day old seedlings of wild type and transgenic plants were germinated and grown on MS medium and then transferred to MS medium containing 350 or 450 mM mannitol. The stressed seedlings were transferred to pots to observe their recovery after stress treatment. A total of 20 seedlings were observed for each of five experiments. Stressed transgenic seedlings showed more yellow cotyledons and/or leaves as compared to wild type seedlings, indicating that GmWRKY13 transgenic plants are more sensitive to osmotic stress treatment under these conditions.

Seven day old seedlings of wild-type and transgenic plants were germinated and grown for 5 days on ABA-free MS medium. The seedlings were then grown on vertically placed plates supplemented with 0, 5, 10, or 20 μM ABA, and the root length was measured after 16 days. On ABA treatment, the root growth of wild type seedlings was severely inhibited, whereas that of transgenic plants was only slightly affected. These results indicate that GmWRKY13 transgenic plants are less sensitive than wild type plants to ABA treatment under these conditions.

### Example 13

### Expression of GmWRKY13 Target Genes

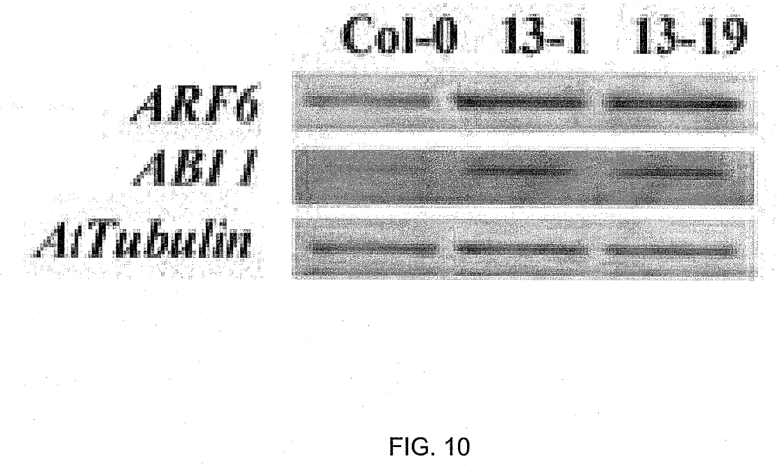

RNA samples were isolated from untreated 2 week old seedlings of wild type plants (Col-0) and GmWRKY13 transgenic lines 13-1 and 13-19. A number of genes that contain four or more WRKY domain-binding sites in their promoter regions, together with several stress-related genes, were selected as candidate target genes for RT-PCR analysis (Liu et al., Plant Cell, 1998, 10: 1391-1406). The tubulin gene was used as an internal control. The expression of ARF6 and ABI1 was up-regulated in GmWRKY13 transgenic plants compared to wild type plants (FIG. 10).

### Example 14

### Cloning of GmWRKY21 cDNA

A GmWRKY21 cDNA was cloned using RT-PCR essentially as described for GmWRKY54 in Example 1, The following primers were used for GmWRKY21 probe template synthesis:

| | |
|---|---|
| 5′-ATGGATTACTATTTTGGAAA-3′ | (SEQ ID NO: 16) |
| 5′-TCATGAGTTTGCAGAAGGGT-3′. | (SEQ ID NO: 17) |

The recovered open reading frame of GmWRKY21 has a nucleotide sequence set forth as SEQ ID NO: 18 and encodes a polypeptide with an amino acid sequence set forth as SEQ ID NO: 22.

### Example 15

### Expression of GmWRKY21 Under Environmental Stress Conditions

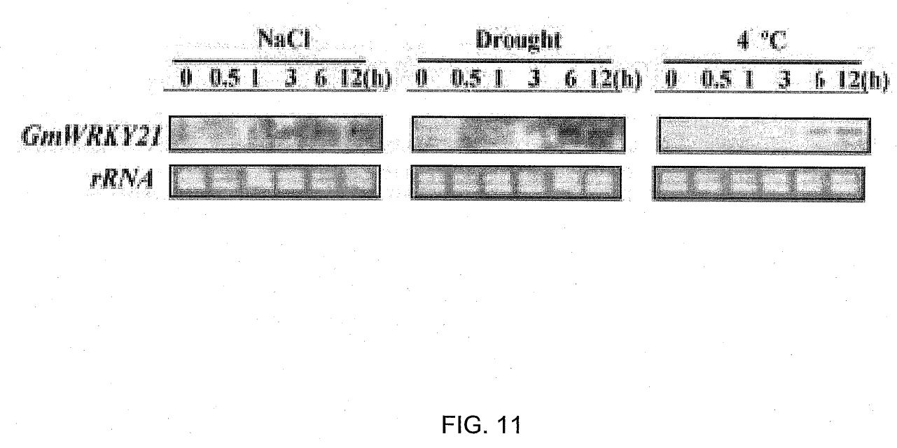

GmWRKY21 expression under environmental stress conditions was assessed essentially as described for GmWRKY54 in Example 2, Total RNA was analyzed by Northern hybridization using GmWRKY21 DNA as a probe. As shown in FIG. 11, the expression of GmWRKY21 was significantly induced by the stress of salt, drought, or low temperature (4° C.).

### Example 16

### Construction of a GmWRKY21 Expression Vector pBin438-GmWRKY21

The cDNA obtained by reverse transcription of total RNA from (Glycine max L.) cv. Kefeng No. 1 was PCR amplified with the following primers specific for GmWRKY21 (SEQ ID NO: 18) and also containing BamHI and KpnI linker sequences:

(SEQ ID NO: 19)

5′-ATCAGGATCCATGGATTACTATTTTGGAAAC-3′

(SEQ ID NO: 20)

5′-GATGGGTACCTCATGAGTTTGCAGAAGGGTG-3′.

The amplified product was digested with BamHI and KpnI, recovered, and inserted between the BamHI and KpnI restriction sites in the sense orientation downstream of the CaMV 35S promoter in the plant binary expression vector pBin438 (Tai-yuan Li, et al., J. Science in China (Column B), 1994, 24(3): 276-282). The resulting vector was designated as pBin438-GmWRKY21.

### Example 17

### Preparation of GmWRKY21 Transgenic Plants

Arabidopsis thaliana were transformed using pBin438-GmWRKY21 essentially as described in Example 4. PCR was performed to identify GmWRKY21 transgenic plants of the T₀ generation using the following primers:

(SEQ ID NO: 19)

5′-ATCAGGATCCATGGATTACTATTTTGGAAAC-3′

(SEQ ID NO: 20)

5′-GATGGGTACCTCATGAGTTTGCAGAAGGGTG-3′.

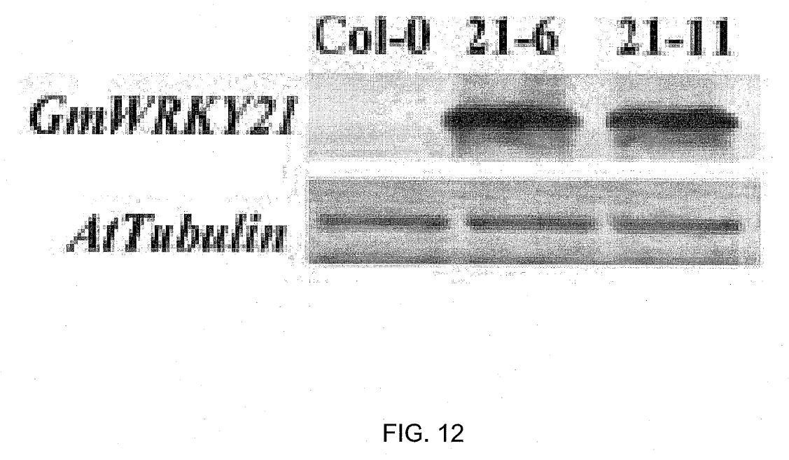

Positive plants of the T₀ generation were subjected to repeated selfing and selection until a T₃ generation was obtained. Expression of GmWRKY21 was verified by Northern hybridization using the GmWRKY21 gene (SEQ ID NO: 18) as a probe. Tubulin expression was used as an internal control. GmWRKY21 was highly expressed in two GmWRKY21 transgenic plants of T₃ generation (21-6 and 21-11), and was expressed at an undetectable level in control plants transformed by blank vector (FIG. 12).

### Example 18

### Freezing Stress Tolerance of GmWRKY21 Transgenic Plants

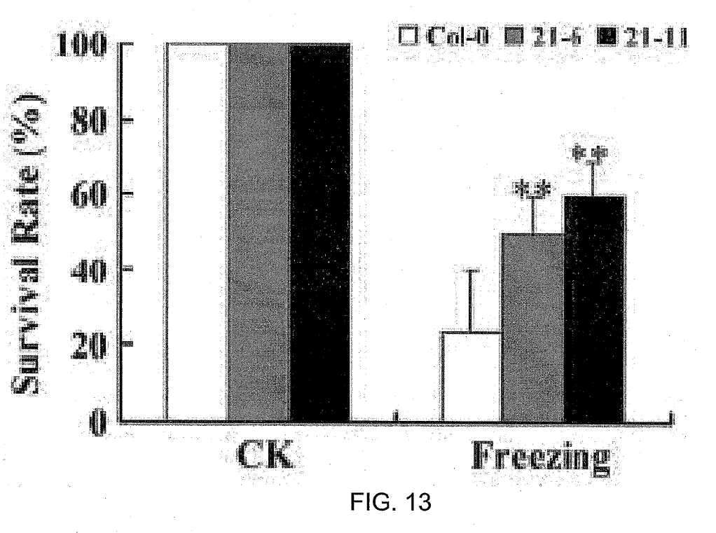

Three week old seedlings of the GmWRKY21 transgenic plant strains 21-6 and 21-11 of the T3 generation and control plants transformed by blank vector were exposed to −20° C. for 80 minutes and then transferred to 22° C. and normal conditions for recovery. The experiment was performed in triplicate with 42 plants for each strain. Plants were observed at 6 and 16 days recovery. The survival rate of the plants exposed to the freezing stress is shown in FIG. 13.

Approximately 24% of the wild type plants survived after freezing treatment (FIG. 13). The transgenic plants exhibited enhanced tolerance to freezing stress, with survival rates of approximately 50% for transgenic line 21-6 and approximately 60% for line 21-11 (FIG. 13).

Five day old seedlings of wild type and transgenic plants were also tested for ABA sensitivity. Seedlings were germinated and grown for 5 days on ABA-free MS medium and then grown on vertically placed plates supplemented with 0, 5, 10, or 20 μM ABA. Root length was measured after 16 days. Relative growth was comparable for wild type and GmWRKY21 transgenic plants at the ABA concentrations tested.

1. An isolated WRKY transcription factor nucleic acid comprising
(a) a nucleotide sequence set forth as SEQ ID NO: 1;
(b) a nucleic acid that specifically hybridizes to the complement of SEQ ID NO: 1 under stringent hybridization conditions;
(c) a nucleotide sequence encoding a WRKY transcription factor protein comprising an amino acid sequence set forth as SEQ ID NO: 2;
(d) a nucleotide sequence set forth as SEQ ID NO: 13;
(e) a nucleic acid that specifically hybridizes to the complement of SEQ ID NO: 13 under stringent hybridization conditions;
(f) a nucleotide sequence encoding a WRKY transcription factor protein comprising an amino acid sequence set forth as SEQ ID NO: 21;
(g) a nucleotide sequence set forth as SEQ ID NO: 18;
(h) a nucleic acid that specifically hybridizes to the complement of SEQ ID NO: 18 under stringent hybridization conditions;
(i) a nucleotide sequence encoding a WRKY transcription factor protein comprising an amino acid sequence set forth as SEQ ID NO: 22;
(j) a nucleotide sequence complementary to that of a nucleic acid of (a)-(i); or
(k) a functional fragment of a nucleic acid of (a)-(j).

2. A vector comprising the nucleic acid of claim 1.

3. A host cell which expresses the vector of claim 2.

4. The host cell of claim 3, which is a plant cell.

5. An isolated WRKY transcription factor protein comprising an amino acid sequence set forth as SEQ ID NO: 2, SEQ ID NO: 21, or SEQ ID NO: 22.

6. An antibody or antibody fragment which specifically binds to the isolated WRKY transcription factor protein of claim 5.

7. A transgenic plant transformed with a nucleic acid of claim 1.

8. The plant of claim 7, which is a monocot.

9. The plant of claim 7, which is a dicot.

10. A method of producing a transgenic plant comprising introducing into a plant cell a recombinant nucleic acid, wherein the recombinant nucleic acid comprises:
(a) a nucleotide sequence set forth as SEQ ID NO: 1;
(b) a nucleic acid that specifically hybridizes to the complement of SEQ ID NO: 1 under stringent hybridization conditions;
(c) a nucleotide sequence encoding a WRKY transcription factor protein comprising an amino acid sequence set forth as SEQ ID NO: 2;
(d) a nucleotide sequence set forth as SEQ ID NO: 13;
(e) a nucleic acid that specifically hybridizes to the complement of SEQ ID NO: 13 under stringent hybridization conditions;
(f) a nucleotide sequence encoding a WRKY transcription factor protein comprising an amino acid sequence set forth as SEQ ID NO: 21;
(g) a nucleotide sequence set forth as SEQ ID NO: 18;
(h) a nucleic acid that specifically hybridizes to the complement of SEQ ID NO: 18 under stringent hybridization conditions
(i) a nucleotide sequence encoding a WRKY transcription factor protein comprising an amino acid sequence set forth as SEQ ID NO: 22;
(j) a nucleotide sequence complementary to that of a nucleic acid of (a)-(i); or
(k) a functional fragment of a nucleic acid of (a)-(j).

11. The method of claim 10, wherein the plant cell is a monocot plant cell.

12. The method of claim 10, wherein the plant cell is a dicot plant cell.

13. The method of claim 10, wherein the recombinant nucleic acid is introduced into the plant cell using a method selected from the group consisting of microparticle bombardment, Agrobacterium-mediated transformation, and whiskers-mediated transformation.

14. The method of claim 13, wherein the introduction of the recombinant nucleic acid results in constitutive overexpression of the nucleic acid sequence of claim 10.

15. The method of claim 10, wherein the plant shows an increase in abiotic stress tolerance.

16. The method of claim 15, wherein the abiotic stress is increased salt concentration.

17. The method of claim 15, wherein the abiotic stress is decreased water concentration.

18. The method of claim 15, wherein the abiotic stress is cold temperature.

19. The method of claim 10, wherein the plant shows improved growth.

20. A plant produced by the method of claim 10.

21. A method of identifying an agent that enhances the expression level of a nucleic acid encoding a WRKY transcription factor protein comprising:
(a) providing a cell recombinantly expressing a WRKY transcription factor protein of claim 5;
(b) contacting the cell with one or more test agents or a control agent;
(c) assaying expression of a nucleic acid encoding the WRKY transcription factor protein;
(d) selecting a test agent that induces elevated expression of the nucleic acid when contacted with the test agent as compared to the control agent.

22. The method of claim 21, wherein the one or more test agents is a peptide, a protein, an oligomer, a nucleic acid, a small molecule, inorganic chemical, organic chemical, or an antibody.

23. A method of increasing the expression level of a nucleic acid encoding a WRKY transcription factor protein comprising contacting a plant or cell expressing a WRKY transcription factor protein with an agent according to claim 21.

24. A method of identifying a binding partner of a WRKY transcription factor protein, the method comprising the steps of:
(a) providing a WRKY transcription factor protein of claim 5;
(b) contacting the WRKY transcription factor protein with one or more test agents or a control agent under conditions sufficient for binding;
(c) assaying binding of a test agent to the isolated WRKY transcription factor protein; and
(d) selecting a test agent that demonstrates specific binding to the WRKY transcription factor protein.

25. The method of claim 24, wherein the one or more test agents is a peptide, a protein, an oligomer, a nucleic acid, a small molecule, inorganic chemical, organic chemical, or an antibody.

26. A method of identifying a WRKY transcription factor modulator comprising the steps of:
(a) providing a cell recombinantly expressing a WRKY transcription factor protein of claim 5;
(b) contacting the cell with one or more test agents or a control agent;
(c) assaying expression of a WRKY transcription factor target gene; and
(d) selecting a test agent that induces a change in expression of the target gene, which gene is normally subject to WRKY transcription factor control, when contacted with the test agent as compared to the control agent.

27. The method of claim 26, wherein the change in expression is an increase in expression.

28. The method of claim 27, wherein the target gene is DREB2A, ERD10, RD29B, ARF6, or ABI1.

29. The method of claim 26, wherein the change in expression is a decrease in expression.

30. The method of claim 29, wherein the target gene is STZ.

31. The method of claim 26, wherein the one or more test agents is a peptide, a protein, an oligomer, a nucleic acid, a small molecule, inorganic chemical, organic chemical, or an antibody.

32. A method of identifying a WRKY transcription factor modulator comprising the steps of:
(a) providing a plant recombinantly expressing a WRKY transcription factor protein of claim 5;
(b) contacting the plant with one or more test agents or a control agent;
(c) assaying survival of the plants under abiotic stress; and
(d) selecting a test agent that promotes survival of the plants under abiotic stress when in the presence of the test agent as compared to the control agent.

33. The method of claim 32, wherein the one or more test agents is a peptide, a protein, an oligomer, a nucleic acid, a small molecule, inorganic chemical, organic chemical, or an antibody.

34. The method of claim 32, wherein the abiotic stress is increased salt concentration.

35. The method of claim 32, wherein the abiotic stress is decreased water concentration.

36. The method of claim 32, wherein the abiotic stress is cold temperature.

37. A method of identifying a WRKY transcription factor modulator comprising the steps of:
(a) providing a plant recombinantly expressing a WRKY transcription factor protein of claim 5;
(b) contacting the plant with one or more test agents or a control agent;
(c) assaying the number of lateral roots of the plant; and
(d) selecting a test agent that increases the number of lateral roots when in the presence of the test agent as compared to the control agent.

38. A plant expressing a heterologous nucleic acid comprising:
(a) a nucleotide sequence set forth as SEQ ID NO: 1;
(b) a nucleic acid that specifically hybridizes to the complement of SEQ ID NO: 1 under stringent hybridization conditions;
(c) a nucleotide sequence encoding a WRKY transcription factor protein comprising an amino acid sequence set forth as SEQ ID NO: 2;
(d) a nucleotide sequence set forth as SEQ ID NO: 13;
(e) a nucleic acid that specifically hybridizes to the complement of SEQ ID NO: 13 under stringent hybridization conditions;
(f) a nucleotide sequence encoding a WRKY transcription factor protein comprising an amino acid sequence set forth as SEQ ID NO: 21;
(g) a nucleotide sequence set forth as SEQ ID NO: 18;
(h) a nucleic acid that specifically hybridizes to the complement of SEQ ID NO: 18 under stringent hybridization conditions;
(i) a nucleotide sequence encoding a WRKY transcription factor protein comprising an amino acid sequence set forth as SEQ ID NO: 22;
(j) a nucleotide sequence complementary to that of a nucleic acid of (a)-(i); or
(k) a functional fragment of a nucleic acid of (a)-(j).

39. The method of claim 38, wherein the plant cell is a monocot plant cell.

40. The method of claim 38, wherein the plant cell is a dicot plant cell.

41. The plant of claim 38, which shows improved growth when compared to a control plant.

42. The plant of claim 38, which shows improved stress tolerance when compared to a control plant.
